(19) United States
(12) Patent Application Publication
(10) Pub. No.: US 2025/0197439 A1
(43) Pub. Date: Jun. 19, 2025

(54) DECARBOXYLATIVE ACETOXYLATION USING MN(II) OR MN(III) REAGENT FOR SYNTHESIS OF 4'-ACETOXY-NUCLEOSIDE AND USE THEREOF FOR SYNTHESIS OF CORRESPONDING 4'-(DIMETHOXYPHOSPHORYL)METHOXY-NUCLEOTIDE

(71) Applicant: DICERNA PHARMACEUTICALS, INC., Lexington, MA (US)

(72) Inventors: Kurt Edric VAGLE, Longmont, CO (US); Sayantan BHADURI, Longmont, CO (US); Guohan YANG, Mansfield, MA (US)

(73) Assignee: DICERNA PHARMACEUTICALS, INC., Lexington, MA (US)

(21) Appl. No.: 18/847,966

(22) PCT Filed: Mar. 17, 2023
(86) PCT No.: PCT/US2023/015498

§ 371 Date: Sep. 17, 2024

Related U.S. Application Data
(60) Provisional application No. 63/269,552, filed on Mar. 18, 2022.

(51) Int. Cl. C07H 19/073; C07H 1/02

(52) U.S. Cl. CPC C07H 19/073; C07H 1/02

(57) ABSTRACT

The present invention relates to the preparation of nucleic acids, nucleosides, nucleotides, and analogues thereof useful as potent and stable RNA interference agents. The present invention provides improved yields and avoids the use of lead tetraacetate for decarboxylative acetylation which decreases the overall cost of production by removing the need for lead remediation and offers a significant reduction in the environmental costs in production.

### CROSS-REFERENCE TO RELATED APPLICATIONS

The present application claims priority under 35 U.S.C. § 119(e) from U.S. Provisional Application No. 63/269,552, filed Mar. 18, 2022, which is incorporated herein by reference in its entirety.

### TECHNICAL FIELD OF THE INVENTION

The present disclosure relates to a process for preparing a compound comprising an acetoxy group from a compound comprising a carboxylic acid utilizing an oxidative decarboxylation acetylation process.

### BACKGROUND OF THE INVENTION

Lead tetraacetate is a common reagent used for decarboxylative acetylation reactions and is often considered an undesirable contaminant or by product in a synthetic process. Decarboxylative acetylation reactions can be influenced by a wide variety of reagents to produce compounds and intermediates of wide synthetic utility. Transition metal complexes are some of the most common and versatile reagents for this type of transformation, however those most used for this type of transformation are considered to be human toxicants. Among these, lead tetraacetate is one of the most chemically reliable reagents that can be used for a wide variety of complex substrates such as carbohydrates and nucleoside derivatives. However, lead is a highly toxic metal, making it undesirable for use in many chemical applications, such as in the preparation of pharmaceuticals. A key starting material for use in some therapeutic oligonucleotides requires a lengthy, linear synthesis that includes a key decarboxylative acetylation step near the end of the synthesis utilizing lead tetraacetate that suffers from low yields (˜50%) which incurs significant costs for the key raw material and the resultant therapeutic oligonucleotide. The use of lead tetraacetate also imposes significant environmental costs and commercial costs because its use and required clean-up negatively impacts some manufacturing facilities or prevents them from producing this key raw material or therapeutic oligonucleotide in a cost-efficient manner. Finally, the use of lead tetraacetate in the supply chain of therapeutic oligonucleotides requires complicated downstream control strategies for the starting material and therapeutic oligonucleotide to maintain patient safety. Ultimately, a need exists to eliminate lead tetraacetate from chemical applications, such as those used for the manufacturing of therapeutic agents, to solve the aforementioned problems, preferably with reagents that have low inherent human toxicity and minimize environmental impacts and corresponding costs.

### SUMMARY

Provided herein is a process for preparing a compound comprising an acetoxy group from a compound comprising a carboxylic acid utilizing decarboxylative acetylation, wherein the conditions comprise a manganese(II) reagent and an oxidizing agent.

In one embodiment, the present disclosure provides a process for preparing a compound comprising an acetoxy group, wherein the compound comprising an acetoxy group is represented by formula B:

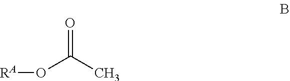

or salt thereof, comprising the steps:
(a) providing a compound comprising a carboxyl group represented by formula A:

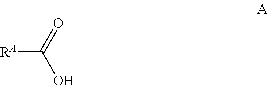

or salt or ester thereof, and
(b) subjecting the compound of formula A to conditions sufficient to form the compound of formula B, wherein the conditions comprise a manganese(II) reagent and an oxidizing agent, and wherein R^{A} is as defined and described herein.

In one embodiment, the present disclosure provides a process for preparing a compound comprising an acetoxy group, wherein the compound comprising an acetoxy group is represented by formula B:

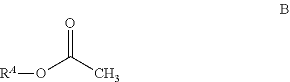

or salt thereof, comprising the steps:
(a) providing a compound comprising a carboxyl group represented by formula A:

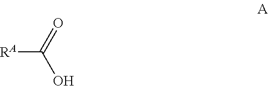

or salt or ester thereof, and
(b) subjecting the compound of formula A to conditions sufficient to form the compound of formula B, wherein the conditions comprise a manganese(III) reagent, and wherein R^{A} is as defined and described herein.

In one embodiment, the present disclosure provides a process for preparing a nucleic acid (e.g., nucleoside) or analogue thereof comprising a 4′-acetoxy group, wherein the nucleoside or analogue thereof comprising a 4′-acetoxy group is represented by formula I-b:

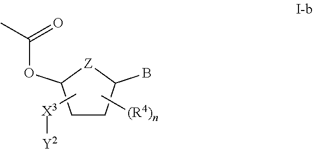

or salt thereof, comprising the steps:
(a) providing a nucleic acid or analogue thereof comprising a 4′-carboxyl group represented by formula I-a:

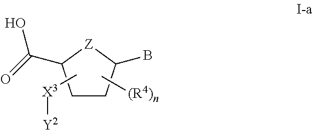

or salt or ester thereof, and
(b) subjecting the nucleic acid or analogue thereof of formula I-a to conditions sufficient to form the nucleoside or analogue thereof of formula I-b, wherein the conditions comprise a manganese(II) reagent and an oxidizing agent, and wherein each variable is as defined and described herein.

In some embodiments, the manganese(II) reagent is Mn(OAc)₂, such as anhydrous Mn(OAc)₂. In some embodiments, the oxidizing agent is (diacetoxyiodo)benzene (DIB). In some embodiments, the conditions further comprise an acid, such as acetic acid. In some embodiments, the conditions further comprise a solvent, such as 1,2-dichloroethane (DCE). In some embodiments, the conditions further comprise heating the reaction mixture to about 20-100° C., about 30-100° C., about 40-100° C., about 50-100° C., about 60-100° C., about 70-100° C., or about 70-90° C. In some embodiments, the conditions further comprise heating the reaction mixture to about 20° C., about 30° C., about 40° C., about 50° C., about 60° C., about 70° C., about 80° C., about 90° C., or about 100° C. In some embodiments, the conditions further comprise heating the reaction mixture for about 6-48 hours, about 12-42 hours, about 18-36 hours, or about 18-30 hours. In some embodiments, the conditions further comprise heating the reaction mixture for about 6 hours, about 12 hours, about 18 hours, about 24 hours, about 30 hours, about 36 hours, about 42 hours, or about 48 hours. In some embodiments, the conditions further comprise heating the reaction mixture to about 80° C. for about 24 hours. In some embodiments, the conditions further comprise heating the reaction mixture for about 2 hours to about 6 hours (e.g., for about 2, 3, 4, 5, or 6 hours).

In one embodiment, the present disclosure provides a process for preparing a nucleic acid (e.g., nucleoside) or analogue thereof comprising a 4′-acetoxy group, wherein the nucleoside or analogue thereof comprising a 4′-acetoxy group is represented by formula I-b:

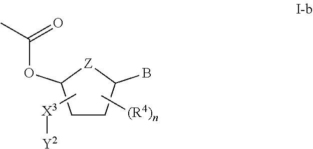

or salt thereof, comprising the steps:
(a) providing a nucleic acid or analogue thereof comprising a 4′-carboxyl group represented by formula I-a:

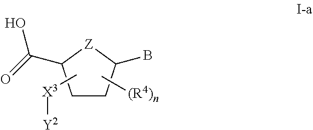

or salt or ester thereof, and
(b) subjecting the nucleic acid or analogue thereof of formula I-a to conditions sufficient to form the nucleoside or analogue thereof of formula I-b, wherein the conditions comprise a manganese(III) reagent, and wherein each variable is as defined and described herein.

In some embodiments, the manganese(III) reagent is Mn(OAc)₃. In some embodiments, a manganese(III) reagent is Mn(OAc)₃ 2H₂O. In some embodiments, a manganese(III) reagent is anhydrous Mn(OAc)₃. In some embodiments, the conditions further comprise an acid, such as acetic acid. In some embodiments, the conditions further comprise a solvent, such as 1,2-dichloroethane (DCE). In some embodiments, the conditions further comprise heating the reaction mixture to about 20-100° C., about 30-100° C., about 40-100° C., about 50-100° C., about 60-100° C., about 70-100° C., or about 70-90° C. In some embodiments, the conditions further comprise heating the reaction mixture to about 20° C., about 30° C., about 40° C., about 50° C., about 60° C., about 70° C., about 80° C., about 90° C., or about 100° C. In some embodiments, the conditions further comprise heating the reaction mixture for about 6-48 hours, about 12-42 hours, about 18-36 hours, or about 18-30 hours. In some embodiments, the conditions further comprise heating the reaction mixture for about 6 hours, about 12 hours, about 18 hours, about 24 hours, about 30 hours, about 36 hours, about 42 hours, or about 48 hours. In some embodiments, the conditions further comprise heating the reaction mixture for about 2 hours to about 6 hours (e.g., for about 2, 3, 4, 5, or 6 hours). In some embodiments, the conditions further comprise heating the reaction mixture to about 80° C. for about 24 hours. In some embodiments, the conditions further comprise heating the reaction mixture to about 80° C. for about 5 hours.

Methods and materials are described herein for use in the present disclosure; other, suitable methods and materials known in the art can also be used. In some aspects, the present disclosure provides an improvement in the art by (a) eliminating the use of toxic lead tetraacetate to achieve decarboxylative acetylation; (b) improving yields over current decarboxylatative acetylations that give products of formula B or I-b; and (c) deceasing the overall cost of production by eliminating the need for lead remediation. The materials, methods, and examples are illustrative only and not intended to be limiting. In case of conflict, the present disclosure, including definitions, will control. Other features and advantages of the present disclosure will be apparent from the following detailed description and from the claims.

### BRIEF DESCRIPTION OF THE FIGURES

FIG. 1 depicts the HPLC chromatogram of the final reaction mixture of reaction #13 shown in Table 7.

### DETAILED DESCRIPTION

### 1. General Description of Certain Embodiments of the Invention

Decarboxylative acetoxylation of carboxylic acid compounds to their corresponding acetoxy compounds is a useful functional group transformation in synthetic chemistry. The present disclosure provides an efficient and facile route to prepare a wide variety of acetoxy compounds from carboxylic acid starting materials. The reaction employs a manganese(II) reagent, such as Mn(OAc)₂, or a manganese(III) reagent, such as Mn(OAc)₃, under mild conditions that is readily transferrable from benchtop to batch process scale-up. The use of a manganese reagent imparts benefits compared to other transition metal complexes because of its low inherent human toxicity, the elimination of complicated downstream control strategies when used in pharmaceutical manufacture and significantly lower environmental impact of waste generated from the process.

4′-O-Methylene phosphonate chemistry for the 5′-terminal phosphate mimic that improves RNAi potency and duration has been described in WO 2018/045317 and U.S. 2019/177729, the entirety of which is herein incorporated by reference. This type of chemical analogue not only mimics the electrostatic and/or steric properties of a phosphate group, but also possesses excellent metabolic stability, and is fully compatible with the standard oligonucleotide solid-phase synthesis. A key building block used in the synthesis of 4′-O-methylene phosphonate containing nucleosides is prepared by a lead tetraacetate promoted decarboxylative acetylation reaction. However, lead is a highly toxic metal, and a very strong poison. In addition, current manufacturing conditions suffer from low yields (˜50-55%) and lead contamination which incurs significant costs for remediation and requires complicated downstream control strategies.

In certain embodiments, the present invention provides improved methods of preparing 4′-O-methylene phosphonate containing nucleic acids and analogues thereof, wherein the improved methods do not use lead tetraacetate for the decarboxylative acetylation reaction. In some embodiments, a provided nucleic acid and analogue thereof of the invention is provided with improved yields over prior methods. In some embodiments, the yield is improved from 50% to 75% yield.

In some embodiments, a provided nucleic acid and analogue thereof of the invention is provided with reduced impurities over prior methods (e.g., ≤1 ppm of lead impurity). In some embodiments, a provided nucleic acid and analogue thereof of the invention is provided with lead impurity that is undetectable by standard detection methods (e.g., ICP-OES). Such improved methods are provided herein for the preparation of nucleic acids and analogues thereof useful as potent and stable RNA interference agents. Such improved methods can be applied to any carboxylic acid to convert them to esters through decarboxylative acetylation

Nucleic acid and analogues thereof of the present disclosure can be used in the preparation of RNA interference agents as well as other chemical synthesis schemes. In some embodiments, RNA interference agents prepared using nucleosides and analogues thereof described herein have the above-mentioned advantages for inhibiting gene expression in a cell.

### 2. Compounds and Definitions

Compounds of the present invention (e.g., nucleic acids and analogues thereof) include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, HANDBOOK OF CHEMISTRY AND PHYSICS, (75^{th} Ed.). Additionally, general principles of organic chemistry are described in ORGANIC CHEMISTRY, (Thomas Sorrell, University Science Books, Sausalito: 1999), and MARCH'S ADVANCED ORGANIC CHEMISTRY, (5^{th} Ed., Ed.: Smith, M. B. and March, J., John Wiley & Sons, New York: 2001), the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclyl group may be monocyclic, bicyclic, bridged bicyclic, or spirocyclic. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic, or heterocyclic, saturated, or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally, or alternatively, any substitutable nitrogen of a bridged bicyclic group is optionally substituted. Exemplary bridged bicyclics include:

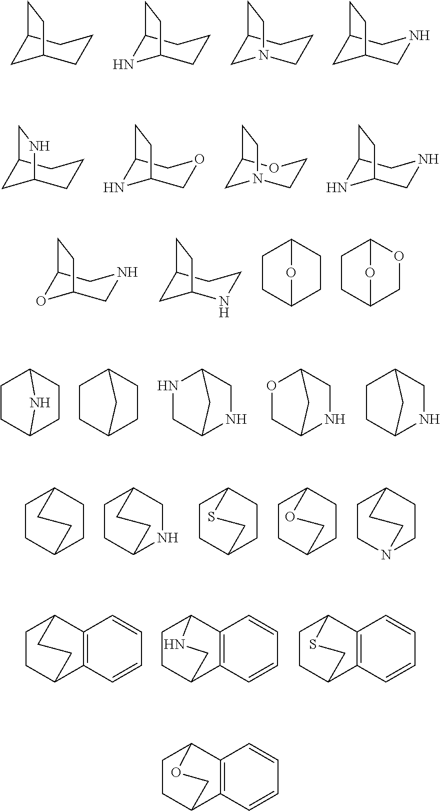

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight, or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

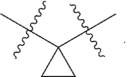 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. In some embodiments, a heterocyclyl group may be monocyclic, bicyclic, bridged bicyclic, or spirocyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═0, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; (CH₂)₀₋₄N(R^{∘})C(O)NR^{∘}₂; —N(R^{∘})C(S)NR^{∘}₂; —(CH₂)₀₋₄N(R^{∘})C(O)OR^{∘}; N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}2; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, (CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and =S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R′)S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR, —NH₂, —NHR^{●}, —NR^{●}2, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the singular forms “a,” “an,” and “the” include plural references unless the context clearly dictates otherwise. For example, a reference to “a method” includes one or more methods, and/or steps of the type described herein and/or which will become apparent to those persons skilled in the art upon reading this disclosure and so forth.

As used herein, the term “and/or” is used in this disclosure to mean either “and” or “or” unless indicated otherwise.

As used herein, the term “4′-O-methylene phosphonate” refers all substituted methylene analogues (e.g., methylene substituted with methyl, dimethyl, ethyl, fluoro, cyclopropyl, etc.) and all phosphonate analogues (e.g., phosphorothioate, phosphorodithiolate, phosphodiester etc.) described herein.

As used herein, the term “5′-terminal nucleotide” refers to the nucleotide located at the 5′-end of an oligonucleotide. The 5′-terminal nucleotide may also be referred to as the “N1 nucleotide” in this application.

As used herein, the term “deoxyribonucleotide” refers to a nucleotide which has a hydrogen group at the 2′-position of the sugar moiety.

As used herein, the term “excipient” refers to a non-therapeutic agent that may be included in a composition, for example to provide or contribute to a desired consistency or stabilizing effect.

As used herein, the term “furanose” refers to a carbohydrate having a five-membered ring structure, where the ring structure has 4 carbon atoms and one oxygen atom represented by

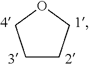

wherein the numbers represent the positions of the 4 carbon atoms in the five-membered ring structure.

As used herein, the term “internucleotide linking group” or “internucleotide linkage” refers to a chemical group capable of covalently linking two nucleoside moieties. Typically, the chemical group is a phosphorus-containing linkage group containing a phospho or phosphite group. Phospho linking groups are meant to include a phosphodiester linkage, a phosphorodithioate linkage, a phosphorothioate linkage, a phosphotriester linkage, a thionoalkylphosphonate linkage, a thionalkylphosphotriester linkage, a phosphoramidite linkage, a phosphonate linkage and/or a boranophosphate linkage. Many phosphorus-containing linkages are well known in the art, as disclosed, for example, in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050. In other embodiments, the oligonucleotide contains one or more internucleotide linking groups that do not contain a phosphorous atom, such short chain alkyl or cycloalkyl internucleotide linkages, mixed heteroatom and alkyl or cycloalkyl internucleotide linkages, or one or more short chain heteroaromatic or heterocyclic internucleotide linkages, including, but not limited to, those having siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; and amide backbones. Non-phosphorous containing linkages are well known in the art, as disclosed, for example, in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

As used herein, the term “modified nucleoside” refers to a nucleoside containing one or more of a modified or universal nucleobase or a modified sugar. The modified or universal nucleobases (also referred to herein as base analogs) are generally located at the 1′-position of a nucleoside sugar moiety and refer to nucleobases other than adenine, guanine, cytosine, thymine, and uracil at the 1′-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). A modified sugar (also referred herein to a sugar analog) includes modified deoxyribose or ribose moieties, e.g., where the modification occurs at the 2′, 3′-, 4′, or 5′-carbon position of the sugar. The modified sugar may also include non-natural alternative carbon structures such as those present in locked nucleic acids (“LNA”) (see, e.g., Koshkin et al. (1998), TETRAHEDRON, 54, 3607-3630); bridged nucleic acids (“BNA”) (see, e.g., U.S. Pat. No. 7,427,672 and Mitsuoka et al. (2009), NUCLEIC ACIDS RES., 37(4):1225-38); and unlocked nucleic acids (“UNA”) (see, e.g., Snead et al. (2013), MOLECULAR THERAPY—NUCLEIC ACIDS, 2, e103 (doi:10.1038/mtna.2013.36)). Suitable modified or universal nucleobases or modified sugars in the context of the present disclosure are described herein.

As used herein, the term “modified nucleotide” refers to a nucleotide containing one or more of a modified or universal nucleobase, a modified sugar, or a modified phosphate. The modified or universal nucleobases (also referred to generally herein as nucleobase) are generally located at the 1′-position of a nucleoside sugar moiety and refer to nucleobases other than adenine, guanine, cytosine, thymine, and uracil at the 1′-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). A modified sugar (also referred herein to a sugar analog) includes modified deoxyribose or ribose moieties, e.g., where the modification occurs at the 2′-, 3′-, 4′-, or 5′-carbon position of the sugar. The modified sugar may also include non-natural alternative carbon structures such as those present in locked nucleic acids (“LNA”) (see, e.g., Koshkin et al. (1998), TETRAHEDRON, 54, 3607-30), bridged nucleic acids (“BNA”) (see, e.g., U.S. Pat. No. 7,427,672 and Mitsuoka et al. (2009), NUCLEIC ACIDS RES., 37(4):1225-38); and unlocked nucleic acids (“UNA”) (see, e.g., Snead et al. (2013), MOLECULAR THERAPY—NUCLEIC ACIDS, 2, e103(doi: 10.1038/mtna.2013.36)). Modified phosphate groups refer to a modification of the phosphate group that does not occur in natural nucleotides and includes non-naturally occurring phosphate mimics as described herein. Modified phosphate groups also include non-naturally occurring internucleotide linking groups, including both phosphorous containing internucleotide linking groups and non-phosphorous containing linking groups, as described herein. Suitable modified or universal nucleobases, modified sugars, or modified phosphates in the context of the present disclosure are described herein.

As used herein, the term “naked nucleic acid” refers to a nucleic acid that is not formulated in a protective lipid nanoparticle or other protective formulation and is thus exposed to the blood and endosomal/lysosomal compartments when administered in vivo.

As used herein, the term “natural nucleoside” refers to a heterocyclic nitrogenous base in N-glycosidic linkage with a sugar (e.g., deoxyribose or ribose or analog thereof). The natural heterocyclic nitrogenous bases include adenine, guanine, cytosine, uracil, and thymine.

As used herein, the term “natural nucleotide” refers to a heterocyclic nitrogenous base in N-glycosidic linkage with a sugar (e.g., ribose or deoxyribose or analog thereof) that is linked to a phosphate group. The natural heterocyclic nitrogenous bases include adenine, guanine, cytosine, uracil, and thymine.

As used herein, the term “nucleic acid or analogue thereof” refers to any natural or modified nucleotide, nucleoside, oligonucleotide, conventional antisense oligonucleotide, ribonucleotide, deoxyribonucleotide, ribozyme, RNAi inhibitor molecule, antisense oligo (ASO), short interfering RNA (siRNA), canonical RNA inhibitor molecule, aptamer, antagomir, exon skipping or splice altering oligos, mRNA, miRNA, or CRISPR nuclease systems comprising one or more of the 4′-O-methylene phosphonate internucleotide linkage described herein. In certain embodiments, the provided nucleic acids or analogues thereof are used in antisense oligonucleotides, siRNA, and dicer substrate siRNA, including those described in U.S. 2010/331389, U.S. Pat. Nos. 8,513,207, 10,131,912, 8,927,705, CA 2,738,625, EP 2,379,083, and EP 3,234,132, the entirety of each of which is herein incorporated by reference. In some embodiments, a nucleic acid refers to a nucleotide or nucleoside. As used herein, the term “nucleic acid inhibitor molecule” refers to an oligonucleotide molecule that reduces or eliminates the expression of a target gene wherein the oligonucleotide molecule contains a region that specifically targets a sequence in the target gene mRNA. Typically, the targeting region of the nucleic acid inhibitor molecule comprises a sequence that is sufficiently complementary to a sequence on the target gene mRNA to direct the effect of the nucleic acid inhibitor molecule to the specified target gene. The nucleic acid inhibitor molecule may include ribonucleotides, deoxyribonucleotides, and/or modified nucleotides.

As used herein, the term “nucleobase” refers to a natural nucleobase, a modified nucleobase, or a universal nucleobase. The nucleobase is the heterocyclic moiety which is located at the 1′ position of a nucleotide sugar moiety in a modified nucleotide that can be incorporated into a nucleic acid duplex (or the equivalent position in a nucleotide sugar moiety substitution that can be incorporated into a nucleic acid duplex). Accordingly, the present invention provides a nucleic acid and analogue thereof comprising a 4′-O-methylene phosphonate internucleotide linkage, wherein the 4′-O-methylene phosphonate internucleotide linkage is represented by formula I where the nucleobase is generally either a purine or pyrimidine base. In some embodiments, the nucleobase can also include the common bases guanine (G), cytosine (C), adenine (A), thymine (T), or uracil (U), or derivatives thereof, such as protected derivatives suitable for use in the preparation of oligonucleotides. In some embodiments, each of nucleobases G, A, and C independently comprises a protecting group selected from isobutyryl, acetyl, difluoroacetyl, trifluoroacetyl, phenoxyacetyl, isopropylphenoxyacetyl, benzoyl, 9-fluorenylmethoxycarbonyl, phenoxyacetyl, dimethylformamidine, dibutylforamidine and N,N-diphenylcarbamate. Nucleobase analogs can duplex with other bases or base analogs in dsRNAs. Nucleobase analogs include those useful in the nucleic acids and analogues thereof and methods of the invention, e.g., those disclosed in U.S. Pat. Nos. 5,432,272 and 6,001,983 to Benner and U.S. Patent Publication No. 20080213891 to Manoharan, which are herein incorporated by reference. Non-limiting examples of nucleobases include hypoxanthine (I), xanthine (X), 3R-D-ribofuranosyl-(2,6-diaminopyrimidine) (K), 3-O-D-ribofuranosyl-(1-methyl-pyrazolo[4,3-d]pyrimidine-5,7(4H,6H)-dione) (P), iso-cytosine (iso-C), iso-guanine (iso-G), 1-β-D-ribofuranosyl-(5-nitroindole), 1-β-D-ribofuranosyl-(3-nitropyrrole), 5-bromouracil, 2-aminopurine, 4-thio-dT, 7-(2-thienyl)-imidazo[4,5-b]pyridine (Ds) and pyrrole-2-carbaldehyde (Pa), 2-amino-6-(2-thienyl)purine (S), 2-oxopyridine (Y), difluorotolyl, 4-fluoro-6-methylbenzimidazole, 4-methylbenzimidazole, 3-methyl isocarbostyrilyl, 5-methyl isocarbostyrilyl, and 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl, and structural derivatives thereof (Schweitzer et al., J. ORG. CHEM., 59:7238-7242 (1994); Berger et al., NUCLEIC ACIDS RESEARCH, 28(15):2911-2914 (2000); Moran et al., J. AM. CHEM. SOC., 119:2056-2057 (1997); Morales et al., J. AM. CHEM. SOC., 121:2323-2324 (1999); Guckian et al., J. AM. CHEM. SOC., 118:8182-8183 (1996); Morales et al., J. AM. CHEM. SOC., 122(6):1001-1007 (2000); McMinn et al., J. AM. CHEM. SOC., 121:11585-11586 (1999); Guckian et al., J. ORG. CHEM., 63:9652-9656 (1998); Moran et al., PROC. NATL. ACAD. SCI., 94:10506-10511 (1997); Das et al., J. CHEM. SOC., PERKIN TRANS., 1:197-206 (2002); Shibata et al., J. CHEM. SOC., Perkin Trans., 1: 1605-1611 (2001); Wu et al., J. AM. CHEM. SOC., 122(32):7621-7632 (2000); O'Neill et al., J. ORG. CHEM., 67:5869-5875 (2002); Chaudhuri et al., J. AM. CHEM. SOC., 117:10434-10442 (1995); and U.S. Pat. No. 6,218,108.). Base analogs may also be a universal base.

As used herein, the term “nucleoside” refers to a natural nucleoside or a modified nucleoside.

As used herein, the term “nucleotide” refers to a natural nucleotide or a modified nucleotide.

As used herein, the term “nucleotide position” refers to a position of a nucleotide in an oligonucleotide as counted from the nucleotide at the 5′-terminus. For example, nucleotide position 1 refers to the 5′-terminal nucleotide of an oligonucleotide.

As used herein, the term “oligonucleotide” as used herein refers to a polymeric form of nucleotides ranging from 2 to 2500 nucleotides. Oligonucleotides may be single-stranded or double-stranded. In certain embodiments, the oligonucleotide has 500-1500 nucleotides, typically, for example, where the oligonucleotide is used in gene therapy. In certain embodiments, the oligonucleotide is single or double stranded and has 7-100 nucleotides. In certain embodiments, the oligonucleotide is single or double stranded and has 15-100 nucleotides. In another embodiment, the oligonucleotide is single or double stranded has 15-50 nucleotides, typically, for example, where the oligonucleotide is a nucleic acid inhibitor molecule. In another embodiment, the oligonucleotide is single or double stranded has 25-40 nucleotides, typically, for example, where the oligonucleotide is a nucleic acid inhibitor molecule. In yet another embodiment, the oligonucleotide is single or double stranded and has 19-40 or 19-25 nucleotides, typically, for example, where the oligonucleotide is a double-stranded nucleic acid inhibitor molecule and forms a duplex of at least 18-25 base pairs. In other embodiments, the oligonucleotide is single stranded and has 15-25 nucleotides, typically, for example, where the oligonucleotide nucleotide is a single stranded RNAi inhibitor molecule. Typically, the oligonucleotide contains one or more phosphorous containing internucleotide linking groups, as described herein. In other embodiments, the internucleotide linking group is a non-phosphorus containing linkage, as described herein.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the nucleic acids and analogues thereof of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium, and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “suitable prodrug” is meant to indicate a compound that may be converted under physiological conditions or by solvolysis to a biologically active nucleic acid or analogue thereof described herein. Thus, the term “prodrug” refers to a precursor of a biologically active nucleic acid or analogue thereof that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., DESIGN OF PRODRUGS (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in BIOREVERSIBLE CARRIERS IN DRUG DESIGN, (ed. Edward B. Roche, American Pharmaceutical Association and Pergamon Press, 1987), both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of suitable prodrugs include, but are not limited to glutathione, acyloxy, thioacyloxy, 2-carboalkoxyethyl, disulfide, thiaminal, and enol ester derivatives of a phosphorus atom-modified nucleic acid. The term “pro-oligonucleotide” or “pronucleotide” or “nucleic acid prodrug” refers to an oligonucleotide which has been modified to be a prodrug of the oligonucleotide. Phosphonate and phosphate prodrugs can be found, for example, in Wiener et al., Prodrugs or phosphonates and phosphates: crossing the membrane, TOP. CURR. CHEM. 2015, 360:115-160, the entirety of which is herein incorporated by reference.

As used herein, the term “phosphoramidite” refers to a nitrogen containing trivalent phosphorus derivative. Examples of suitable phosphoramidites are described herein.

As used herein, the term “protecting group” (“PG”) refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found herein and in Greene and Wuts, Protective Groups in Organic Chemistry, (3rd Ed., 1999, John Wiley & Sons, N.Y). and Harrison et al., Compendium of Synthetic Organic Methods, (Vols. 1-8, 1971-1996, John Wiley & Sons, N.Y). Representative nitrogen protecting groups include, but are not limited to, formyl, acetyl, trifluoroacetyl, benzyl, methoxymethyl (“MOM”), benzyloxycarbonyl (“CBZ”), tert-butoxycarbonyl (“Boc”), trimethylsilyl (“TMS”), 2-trimethylsilyl-ethanesulfonyl (“2-TES”), triethylsilyl (“TES”), triisopropylsilyl (“TIPS”), tert-butyldimethylsilyl (“TBDMS”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl, picolyl and trityl ethers, as well as alkyl ethers, tetrahydropyranyl ethers, trialkylsilyl ethers (e.g., TMS, TES, TIPS, or TBDMS groups), glycol ethers, such as ethylene glycol and propylene glycol derivatives and allyl ethers. Representative carboxylic acid protecting groups include, but are not limited to, optionally substituted C₁₋₆ aliphatic esters, optionally substituted aryl esters, optionally substituted benzyl esters, silyl esters, dihydroxazoles, activated esters (e.g., derivatives of nitrophenol, pentafluorophenol, N-hydroxylsuccinimide, hydroxybenzotriazole, etc.), orthoesters, and the like.

As used herein, the term “provided nucleic acid” refers to any genus, subgenus, and/or species set forth herein.

As used herein, the term “ribonucleotide” refers to a natural or modified nucleotide which has a hydroxyl group at the 2′-position of the sugar moiety.

As used herein, the term “RNAi inhibitor molecule” refers to either (a) a double stranded nucleic acid inhibitor molecule (“dsRNAi inhibitor molecule”) having a sense strand (passenger) and antisense strand (guide), where the antisense strand or part of the antisense strand is used by the Argonaute 2 (Ago2) endonuclease in the cleavage of a target mRNA or (b) a single stranded nucleic acid inhibitor molecule (“ssRNAi inhibitor molecule”) having a single antisense strand, where that antisense strand (or part of that antisense strand) is used by the Ago2 endonuclease in the cleavage of a target mRNA.

As used herein, “universal base” refers to a heterocyclic moiety located at the 1′ position of a nucleotide sugar moiety in a modified nucleotide, or the equivalent position in a nucleotide sugar moiety substitution, that, when present in a nucleic acid duplex, can be positioned opposite more than one type of base without altering the double helical structure (e.g., the structure of the phosphate backbone). Additionally, the universal base does not destroy the ability of the single stranded nucleic acid in which it resides to duplex to a target nucleic acid. The ability of a single stranded nucleic acid containing a universal base to duplex a target nucleic can be assayed by methods apparent to one in the art (e.g., UV absorbance, circular dichroism, gel shift, single stranded nuclease sensitivity, etc.). Additionally, conditions under which duplex formation is observed may be varied to determine duplex stability or formation, e.g., temperature, as melting temperature (Tm) correlates with the stability of nucleic acid duplexes. Compared to a reference single stranded nucleic acid that is exactly complementary to a target nucleic acid, the single stranded nucleic acid containing a universal base forms a duplex with the target nucleic acid that has a lower Tm than a duplex formed with the complementary nucleic acid. However, compared to a reference single stranded nucleic acid in which the universal base has been replaced with a base to generate a single mismatch, the single stranded nucleic acid containing the universal base forms a duplex with the target nucleic acid that has a higher Tm than a duplex formed with the nucleic acid having the mismatched base.

Some universal bases are capable of base pairing by forming hydrogen bonds between the universal base and all of the bases guanine (G), cytosine (C), adenine (A), thymine (T), and uracil (U) under base pair forming conditions. A universal base is not a base that forms a base pair with only one single complementary base. In a duplex, a universal base may form no hydrogen bonds, one hydrogen bond, or more than one hydrogen bond with each of G, C, A, T, and U opposite to it on the opposite strand of a duplex. Preferably, the universal bases do not interact with the base opposite to it on the opposite strand of a duplex. In a duplex, base pairing between a universal base occurs without altering the double helical structure of the phosphate backbone. A universal base may also interact with bases in adjacent nucleotides on the same nucleic acid strand by stacking interactions. Such stacking interactions stabilize the duplex, especially in situations where the universal base does not form any hydrogen bonds with the base positioned opposite to it on the opposite strand of the duplex. Non-limiting examples of universal-binding nucleotides include inosine, 1-β-D-ribo furanosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside, NUCLEIC ACIDS RES. 1995 Nov. 11; 23(21):4363-70; Loakes et al., 3-Nitropyrrole and 5-nitroindole as universal bases in primers for DNA sequencing and PCR, NUCLEIC ACIDS RES. 1995 Jul. 11; 23(13):2361-6; Loakes and Brown, 5-Nitroindole as a universal base analogue, NUCLEIC ACIDS RES. 1994 Oct. 11; 22(20):4039-43).

As used herein, the terms “about” or “approximately”, used in conjunction with a numerical value, refer to a range by extending the boundaries above and below the numerical values. For example, the terms “about” or “approximately” can extend the stated value by a variance of 30%, 25%, 20%, 15%, 10%, 9%, 8%, 7%, 6%, 5%, 4%, 3%, 2%, 1%, or 0.5% up and/or down (higher or lower). In some embodiments, the terms “about” or “approximately” extend the stated value by a variance of 25% up and/or down (higher or lower). In some embodiments, the terms “about” or “approximately” extend the stated value by a variance of 10% up and/or down (higher or lower). In some embodiments, the terms “about” or “approximately” extend the stated value by a variance of 5% up and/or down (higher or lower).

### 3. General Methods of Providing Acetoxy Compounds and Salts Thereof

According to one aspect, the present invention provides a process for preparing a compound comprising an acetoxy group, wherein the compound comprising an acetoxy group is represented by formula B:

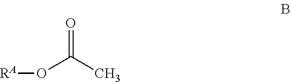

or salt thereof, comprising the steps:
(a) providing a compound comprising a carboxyl group represented by formula A:

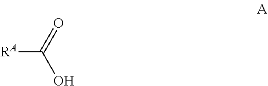

or salt or ester thereof, and
(b) subjecting the compound of formula A to conditions sufficient to form the compound of formula B, wherein the conditions comprise a manganese(II) reagent and an oxidizing agent, and wherein:
R^{A} is an optionally substituted group selected from alkyl, aryl, heteroaryl, carbocyclyl, heterocyclyl, protected amino acid, protected nucleoside, protected nucleotide, and protected oligonucleotide, wherein each of aryl and heteroaryl is independently monocyclic or bicyclic and each of carbocyclyl and heterocyclyl is independently monocyclic, bicyclic, bridged bicyclic, or spirocyclic.

According to one aspect, the present invention provides a process for preparing a compound comprising an acetoxy group, wherein the compound comprising an acetoxy group is represented by formula B:

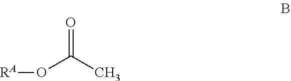

or salt thereof, comprising the steps:
(a) providing a compound comprising a carboxyl group represented by formula A:

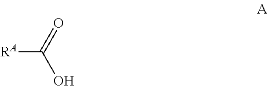

or salt or ester thereof, and
(b) subjecting the compound of formula A to conditions sufficient to form the compound of formula B, wherein the conditions comprise a manganese(III) reagent, and wherein:
   R^{A} is an optionally substituted group selected from alkyl, aryl, heteroaryl, carbocyclyl, heterocyclyl, protected amino acid, protected nucleoside, protected nucleotide, and protected oligonucleotide, wherein each of aryl and heteroaryl is independently monocyclic or bicyclic and each of carbocyclyl and heterocyclyl is independently monocyclic, bicyclic, bridged bicyclic, or spirocyclic.

According to one embodiment, the manganese(II) reagent used in step (b) above is selected from Mn(OAc)₂, MnF₂, MnCl₂, MnBr₂, MnI₂, Mn(NO₂)₂, Mn(ClO₄)₂, MnSO₄, MnCO₃, manganese(II) formate, manganese(II) acetylacetonate, manganese(II) propionate, manganese(II) butyrate, manganese(II) cyclohexane butyrate, and manganese(II) tartrate. In certain embodiments, the manganese(II) reagent is Mn(OAc)₂. In certain embodiments, the manganese(II) reagent is anhydrous Mn(OAc)₂. In some embodiments, the amount of manganese(II) reagent used in step (b) above is about 0.5 molar equivalents to about 2 molar equivalents (e.g., about 0.8, 0.9, 1.0, 1.1, 1.2, 1.3, 1.4, or 1.5 molar equivalents) to the compound of formula A or salt thereof. In certain embodiments, about 1 molar equivalent of manganese(II) reagent (e.g., Mn(OAc)₂) is used. In some embodiments, the manganese(II) reagent and amount used is as depicted in the Examples section.

According to one embodiment, the manganese(III) reagent used in step (b) above is selected from Mn(OAc)₃, MnF₃, MnCl₃, MnBr₃, MnI₃, Mn(NO₂)₃, Mn(ClO₄)₃, (Mn)₃(SO₄)₂, (Mn)₃(CO₃)₂, manganese(III) formate, manganese(III) acetylacetonate, manganese(III) propionate, manganese(III) butyrate, manganese(III) cyclohexane butyrate, and manganese(III) tartrate. In certain embodiments, the manganese(III) reagent is Mn(OAc)₃. In certain embodiments, the manganese(III) reagent is anhydrous Mn(OAc)₃. In some embodiments, the amount of manganese(III) reagent used in step (b) above is about 0.5 molar equivalents to about 2 molar equivalents (e.g., about 0.8, 0.9, 1.0, 1.1, 1.2, 1.3, 1.4, or 1.5 molar equivalents) to the compound of formula A or salt thereof. In certain embodiments, about 1 molar equivalent of manganese(III) reagent (e.g., Mn(OAc)₃) is used. In some embodiments, the manganese(III) reagent and amount used is as depicted in the Examples section.

According to another embodiment, the oxidizing reagent in step (b) above is selected from a mixture of elemental iodine and hydrogen peroxide, hypervalent iodine reagents (e.g., (diacetoxyiodo)benzene, bis(trifluoroacetate)iodobenzene, Togni's reagent, etc.), urea hydrogen peroxide complex, silver nitrate/silver sulfate, sodium bromate, ammonium peroxydisulfate, tetrabutylammonium peroxydisulfate, potassium persulfate, Oxone®, Chloramine T, Selectfluor®, Selectfluor® II, sodium hypochlorite, potassium iodate/sodium periodate, N-iodosuccinimide, N-bromosuccinimide, N-chlorosuccinimide, 1,3-diiodo-5,5-dimethylhydantion, pyridinium tribromide, and iodine monochloride, m-chloroperoxybenzoic acid or complexes thereof. In certain embodiments, the oxidizing agent is (diacetoxyiodo)benzene (DIB). In some embodiments, the amount of oxidizing reagent used in step (b) above is about 1 molar equivalents to about 3 molar equivalents (e.g., about 1.0, 1.1, 1.2, 1.3, 1.4, 1.5, 1.6, 1.7, 1.8, 1.9, or 2.0 molar equivalents) to compound of formula A or salt thereof. In certain embodiments, about 1.5 molar equivalent of oxidizing reagent (e.g., DIB) is used. In some embodiments, the oxidizing reagent and amount used is as depicted in the Examples section.

According to another embodiment, the conditions used in step (b) above may also include an acid. In some embodiments, the acid an inorganic acid (e.g., hydrochloric acid, phosphoric acid, sulfuric acid, etc.) or an organic acid (e.g., acetic acid, trifluoroacetetic acid, methansulfonic acid, para-toluenesulfonic acid, etc.). In certain embodiments, the acid is acetic acid (AcOH). In some embodiments, the amount of acid used in step (b) above is about 0.5 molar equivalents to about 2 molar equivalents (e.g., about 0.8, 0.9, 1.0, 1.1, 1.2, 1.3, 1.4, or 1.5 molar equivalents) to compound of formula A or salt thereof. In certain embodiments, about 1 molar equivalent of acid (e.g., AcOH) is used. In some embodiments, the acid and amount used is as depicted in the Examples section.

According to another embodiment, the conditions used in step (b) above may also include an acetate source. In some embodiments, the acetate source is any organic or inorganic compound that may provide an acetate ion (e.g., AcO⁻) for reaction (e.g., acetic acid, sodium acetate, metal acetates, etc.). In certain embodiments, the acetate source is acetic acid (AcOH). In some embodiments, the amount of acetate source used in step (b) above is about 0.5 molar equivalents to about 2 molar equivalents (e.g., about 0.8, 0.9, 1.0, 1.1, 1.2, 1.3, 1.4, or 1.5 molar equivalents) to compound of formula A or salt thereof. In certain embodiments, about 1 molar equivalent of acetate source (e.g., AcOH) is used. In some embodiments, the acetate source and amount used is as depicted in the Examples section.

According to another embodiment, the conditions used in step (b) above may also include a solvent. In some embodiments, the solvent is selected from water, alcohols (e.g., methanol, ethanol, isopropanol, etc.), ethers (diethyl ether, tetrahydrofuran, 2-methyltetrahydrofuran, dioxane, etc.), esters (ethyl acetate, isopropyl acetate, etc.), ketones (e.g., acetone, etc.), halocarbons (dichloromethane, 1,2-dichloroethane, etc.), aromatic hydrocarbons (toluene, xylenes, etc.), N,N-dimethylformamide, N,N-dimethylacetamide, dimethyl sulfoxide, and mixtures thereof. In certain embodiments, the solvent is 1,2-dichloroethane (DCE). In some embodiments, the volume (V) of solvent used in step (b) above is about 5 V to about 15 V (e.g., about 6, 7, 8, 9, 10, 11, 12, 13, or 14 V); wherein Volume (V) is 1 mL of solvent per gram of substrate. In certain embodiments, about 1 V of solvent (e.g., DCE) is used. In some embodiments, the solvent and volume (V) used is as depicted in the Examples section.

According to another embodiment, the conditions used in step (b) above may also include heating the reaction to a temperature for an amount of time. In some embodiments, the heating comprises a temperature of about room temperature (e.g., 20° C.) to about 100° C. (e.g., about 30, 40, 50, 60, 70, 80, or 90° C.) for about 6 hours to about 48 hours (e.g., for about 8, 10, 12, 14, 16, 18, 20, 22, 24, 26, 28, 30, or 32 hours). In some embodiments, the conditions further comprise heating the reaction mixture for about 2 hours to about 6 hours (e.g., for about 2, 3, 4, 5, or 6 hours). In some embodiments, the conditions comprise heating the reaction to about 80° C. for about 24 hours. In some embodiments, the conditions further comprise heating the reaction mixture to about 80° C. for about 5 hours. In some embodiments, the reaction temperature and duration used is as depicted in the Examples section.

As defined above and described herein, R^{A} is an optionally substituted group selected from alkyl, aryl, heteroaryl, carbocyclyl, heterocyclyl, protected amino acid, protected nucleoside, protected nucleotide, and protected oligonucleotide, wherein each of aryl and heteroaryl is independently monocyclic or bicyclic and each of carbocyclyl and heterocyclyl is independently monocyclic, bicyclic, bridged bicyclic, or spirocyclic.

In some embodiments, R^{A} is an optionally substituted group selected from aryl, heteroaryl, protected nucleoside or protected nucleotide.

In some embodiments, R^{A} is an optionally substituted alkyl (e.g., straight chain or branched C₃₋₁₂ alkyl). In some embodiments, R^{A} is an optionally substituted aryl (e.g., phenyl, naphthyl, etc.). In some embodiments, R^{A} is an optionally substituted heteroaryl (e.g., pyrrolyl, pyrazolyl, indolizinyl, etc.). In some embodiments, R^{A} is an optionally substituted carbocyclyl (e.g., C₃_₆ carbocycle, etc.). In some embodiments, R^{A} is an optionally substituted heterocyclyl (e.g., pyrrolidinyl, piperidinyl, morpholinyl, etc.). In some embodiments, R^{A} is an optionally substituted bridged carbocyclic (e.g., bicyclo[2.2.1]heptane, etc.). In some embodiments, R^{A} is an optionally substituted bridged heterocyclic (e.g., 1-azabicyclo[3.2.1]octane, etc.). In some embodiments, R^{A} is an optionally substituted bicyclic carbocyclic (e.g., octahydro-1H-indene, etc.). In some embodiments, R^{A} is an optionally substituted bicyclic heterocyclic (e.g., indolinyl, octahydroindolizinyl, etc.). In some embodiments, R^{A} is an optionally substituted unprotected amino acid (e.g., alanine, valine, etc.). In some embodiments, R^{A} is an optionally substituted protected amino acid (e.g., N-Boc-alanine, N-Boc-valine, etc.). In some embodiments, R^{A} is an optionally substituted unprotected nucleoside (e.g., natural nucleoside or modified nucleoside as defined herein). In some embodiments, R^{A} is an optionally substituted protected nucleoside (e.g., natural nucleoside or modified nucleoside as defined herein). In some embodiments, R^{A} is an optionally substituted unprotected nucleotide (e.g., natural nucleotide or modified nucleotide as defined herein). In some embodiments, R^{A} is an optionally substituted protected nucleotide (e.g., natural nucleotide or modified nucleotide as defined herein).

In some embodiments, R^{A} is an unprotected nucleoside selected from a 4′-acetoxy derivative of 2′-deoxy-2′-fluorouridine (fU), 2′-O-methyluridine (mU), 2′-deoxy-2′-fluorouguanosine (fG), 2′-O-methylguanosine (mG), 2′-deoxy-2′-fluoroadenosine (fA), 2′-O-methyladenosine (mA), 2′-O-methylcytidine (mC), and 2′-deoxy-2′-fluorocytidine (fC).

In some embodiments, R^{A} is a protected nucleoside selected from a 4′-acetoxy derivative of 2′-deoxy-2′-fluorouridine (fU), 2′-O-methyluridine (mU), 2′-deoxy-2′-fluorouguanosine (fG), 2′-O-methylguanosine (mG), 2′-deoxy-2′-fluoroadenosine (fA), 2′-O-methyladenosine (mA), 2′-O-methylcytidine (mC), and 2′-deoxy-2′-fluorocytidine (fC), wherein each of the nucleobases of fU, mU, fG, mG, fA, mA, mC, and fC independently comprises a protecting group and/or each of the 3′-hydroxy groups are protected with a suitable hydroxyl protecting group.

In some embodiments, the optionally substituted group of R^{A} is selected from, but not limited to, C₁₋₆ aliphatic, phenyl, a 4-7 membered saturated or partially unsaturated carbocyclic or heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, halogen (e.g., F, Cl, Br, I), —CN, —NO₂, —OH, —OC₁₋₆alkyl, —SH, —SC₁₋₆alkyl, —NH₂, —NHC₁₋₆alkyl, —N(C₁₋₆alkyl)₂, —SO₂C₁₋₆alkyl, —SO₂NH₂, —SO₂NHC₁₋₆alkyl, —SO₂N(C₁₋₆alkyl)₂, —S(O)C₁₋₆alkyl, —CF(C₁₋₆alkyl)₂, —CF₂H, —CF₂C₁₋₆alkyl, —CF₃, —C(C₀₋₆alkyl)₂OC₀₋₆alkyl, —C(C₀₋₆alkyl)₂N(C₀₋₆alkyl)₂, —C(O)C₁₋₆alkyl, —CO₂C₁₋₆alkyl, —C(O)N(C₁₋₆alkyl)₂, —OC(O)C₁₋₆alkyl, —OC(O)N(C₁₋₆alkyl)₂, —NHCO₂C₁₋₆alkyl, —NHC(O)C₁₋₆alkyl, and —NHSO₂C₁₋₆alkyl.

In some embodiments, the optionally substituted group of R^{A} is described in the definitions section herein.

In some embodiments, R^{A} is pentyl. In some embodiments, R^{A} is

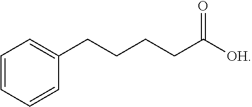 [SMILES: O=C(O)CCCCc1ccccc1]

In some embodiments, R^{A} is

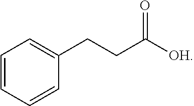 [SMILES: O=C(O)CCc1ccccc1]

In some embodiments, R^{A} is

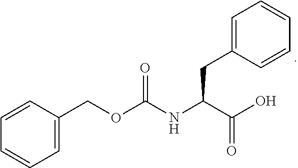 [SMILES: O=C(N[C@@H](Cc1ccccc1)C(=O)O)OCc1ccccc1]

In some embodiments, R^{A} is N-Boc-amino acid. In some embodiments, R^{A} is

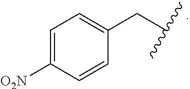 [SMILES: *Cc1ccc([N+](=O)[O-])cc1]

In some embodiments, R^{A} is

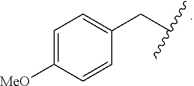 [SMILES: *Cc1ccc(OC)cc1]

In some embodiments, R^{A} is

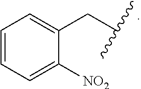 [SMILES: *Cc1ccccc1[N+](=O)[O-]]

In some embodiments, R^{A} is

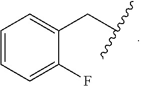 [SMILES: *Cc1ccccc1F]

In some embodiments, R^{A} is

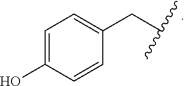 [SMILES: *Cc1ccc(O)cc1]

In some embodiments, R^{A} is

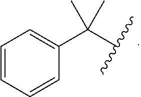 [SMILES: *C(C)(C)c1ccccc1]

In some embodiments, R^{A} is

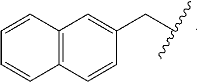 [SMILES: *Cc1ccc2ccccc2c1]

In some embodiments, R^{A} is

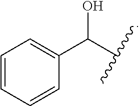 [SMILES: *C(O)c1ccccc1]

In some embodiments, R^{A} is

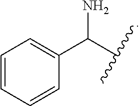 [SMILES: *C(N)c1ccccc1]

In some embodiments, R^{A} is

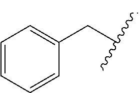 [SMILES: *Cc1ccccc1]

In some embodiments, R^{A} is

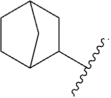 [SMILES: *C1CC2CCC1C2]

In some embodiments, R^{A}

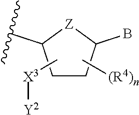

as represented by formula I-a described herein.

### 4. General Methods of Providing the Nucleosides and Analogues Thereof

The schemes provided herein are merely illustrative of some methods by which the compounds of the present disclosure can be synthesized, and various modifications of these schemes can be made and suggested by those skilled in the art having referred to this disclosure.

In Scheme A below, where a particular protecting group, leaving group, or transformation condition is depicted, one of ordinary skill in the art will appreciate that other protecting groups, leaving groups, and transformation conditions are also suitable and are contemplated. Certain reactive functional groups (e.g., —N(H)—, —OH, etc.) envisioned in the genera in Scheme A requiring additional protection group strategies are also contemplated and is appreciated by those having ordinary skill in the art. Such groups and transformations are described in detail in MARCH'S ADVANCED ORGANIC CHEMISTRY: Reactions, Mechanisms, and Structure, (M. B. Smith and J. March, 5^{th} Edition, John Wiley & Sons, 2001), COMPREHENSIVE ORGANIC TRANSFORMATIONS, (R. C. Larock, 2^{nd} Edition, John Wiley & Sons, 1999), and PROTECTING GROUPS IN ORGANIC SYNTHESIS, (T. W. Greene and P. G. M. Wuts, 3^{rd} edition, John Wiley & Sons, 1999), the entirety of each of which is hereby incorporated herein by reference.

In certain embodiments, nucleic acids, and analogues thereof of the present invention are generally prepared according to Scheme A set forth below:

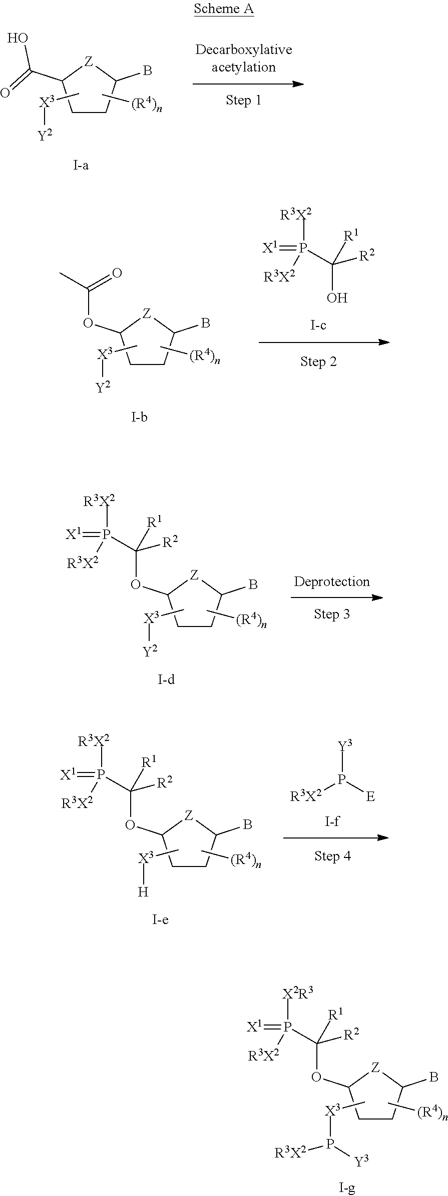

As depicted in Scheme A above, a nucleoside or analogue thereof of formula I-a or salt thereof comprising a 4′-acetoxy group is subjected to decarboxylative acetylation conditions using a manganese(II) reagent and an oxidizing agent to form a nucleoside or analogue thereof of formula I-b or salt thereof (Step 1). In some embodiments, the decarboxylative acetylation conditions in Step 1 comprise a manganese(III) reagent. Nucleoside or analogue thereof of formula I-b or salt thereof is then reacted with a compound of formula I-c or salt thereof to form a nucleotide or analogue thereof of formula I-d or salt thereof (Step 2). Nucleotide or analogue thereof of formula I-d or salt thereof is then subjected to deprotection conditions to form a nucleotide or analogue thereof of formula I-e or salt thereof (Step 3). In some embodiments, the deprotection conditions in Step 3 remove the protecting group Y² and any protecting groups on nucleobase B. Nucleotide or analogue thereof of formula I-e or salt thereof is then reacted with a compound of formula I-f or salt thereof (e.g., a P(III) reagent) to form a nucleotide or analogue thereof of formula I-g or salt thereof (Step 4). Each of B, E, R¹, R², R³, R⁴, X¹, X², X³, Y², Y³, Z, and n is as defined and described herein.

In some embodiments, one or more of a nucleoside, nucleotide, or analogue thereof of formula I-b, I-d, I-e, or I-g, or salt thereof has ≤1 ppm of lead impurity as measured by ICP-OES. In some embodiments, a nucleoside or analogue thereof of formula I-b or salt thereof has ≤1 ppm of lead impurity as measured by ICP-OES. In some embodiments, a nucleoside, nucleotide, or analogue thereof of formula I-d or salt thereof has ≤1 ppm of lead impurity as measured by ICP-OES. In some embodiments, a nucleoside, nucleotide, or analogue thereof of formula I-e or salt thereof has ≤1 ppm of lead impurity as measured by ICP-OES. In some embodiments, a nucleoside, nucleotide, or analogue thereof of formula I-g or salt thereof has ≤1 ppm of lead impurity as measured by ICP-OES.

One of skill in the art will appreciate that various functional groups present in the nucleosides, nucleotides, or analogues thereof of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See for example, MARCH'S ADVANCED ORGANIC CHEMISTRY, (5^{th} Ed., Ed.: Smith, M. B. and March, J., John Wiley & Sons, New York: 2001), the entirety of each of which is herein incorporated by reference. Such interconversions may require one or more of the aforementioned techniques, and certain methods for synthesizing the provided nucleosides and nucleotides of the invention are described below in the Exemplification.

According to one aspect, the present invention provides a process for preparing a nucleoside or analogue thereof comprising a 4′-acetoxy group, wherein the nucleoside or analogue thereof comprising a 4′-acetoxy group is represented by formula I-b:

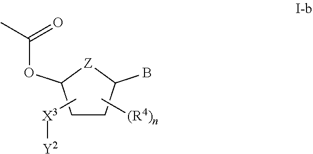

or a salt thereof, comprising the steps:
(a) providing a nucleoside or analogue thereof comprising a 4′-carboxyl group represented by formula I-a:

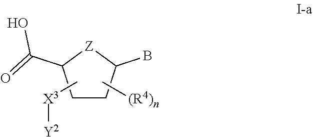

or a salt or ester thereof, and
(b) subjecting the nucleoside or analogue thereof of formula I-a to conditions sufficient to form the nucleoside or analogue thereof of formula I-b,
wherein the conditions comprise a manganese(II) reagent and an oxidizing agent, and wherein:
each B is independently a nucleobase or hydrogen;
each R⁴ is independently hydrogen, fluoro, —OH, —OC₁₋₆alkyl, —OCH₂CH₂OC₁₋₆alkyl, or —O-protecting group (—OPG);
each X³ is independently —O—, —S—, or —N(R)—;
each R is independently hydrogen, a protecting group (PG), or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur;
each Y² is independently hydrogen or a protecting group (PG);
each Z is independently —O—, —S—, —N(R)—, or —C(R)₂—; and
each n is independently 0, 1, 2, 3, 4, or 5.

According to one aspect, the present invention provides a process for preparing a nucleoside or analogue thereof comprising a 4′-acetoxy group, wherein the nucleoside or analogue thereof comprising a 4′-acetoxy group is represented by formula I-b:

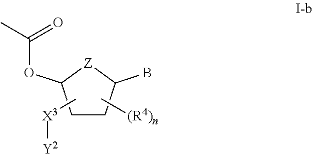

or a salt thereof, comprising the steps:
(a) providing a nucleoside or analogue thereof comprising a 4′-carboxyl group represented by formula I-a:

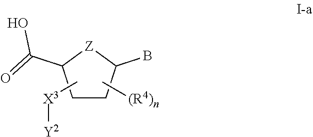

or a salt or ester thereof, and
(b) subjecting the nucleoside or analogue thereof of formula I-a to conditions sufficient to form the nucleoside or analogue thereof of formula I-b,
wherein the conditions comprise a manganese(III) reagent, and wherein:
each B is independently a nucleobase or hydrogen;
each R⁴ is independently hydrogen, fluoro, —OH, —OC₁₋₆alkyl, —OCH₂CH₂OC₁₋₆alkyl, or —O-protecting group (—OPG);
each X³ is independently —O—, —S—, or —N(R)—;
each R is independently hydrogen, a protecting group (PG), or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur;
each Y² is independently hydrogen or a protecting group (PG);
each Z is independently —O—, —S—, —N(R)—, or —C(R)₂—; and
each n is independently 0, 1, 2, 3, 4, or 5.

In some embodiments, a manganese(II) reagent used in step (b) above (or Step (1) of Scheme A) is selected from the group consisting of Mn(OAc)₂, MnF₂, MnCl₂, MnBr₂, MnI₂, Mn(NO₂)₂, Mn(ClO₄)₂, MnSO₄, MnCO₃, MnSO₄, manganese(II) formate, manganese(II) acetylacetonate, manganese(II) propionate, manganese(II) butyrate, manganese(II) cyclohexane butyrate, and manganese(II) tartrate. In certain embodiments, the manganese(II) reagent is Mn(OAc)₂. In certain embodiments, the manganese(II) reagent is anhydrous Mn(OAc)₂. In some embodiments, the amount of manganese(II) reagent used in step (b) above (or Step (1) of Scheme A) is about 0.5 molar equivalents to about 2 molar equivalents (e.g., about 0.8, 0.9, 1.0, 1.1, 1.2, 1.3, 1.4, or 1.5 molar equivalents) to the nucleoside or analogue thereof of formula I-a or salt thereof. In certain embodiments, about 1 molar equivalent of a manganese(II) reagent (e.g., Mn(OAc)₂) is used. In some embodiments, the manganese(II) reagent and amount used is as depicted in the Examples section.

In some embodiments, a manganese(III) reagent used in step (b) above (or Step (1) of Scheme A) is selected from the group consisting of Mn(OAc)₃, MnF₃, MnCl₃, MnBr₃, MnI₃, Mn(NO₂)₃, Mn(ClO₄)₃, (Mn)₂(SO₄)₃, (Mn)₂(CO₃)₃, manganese(III) formate, manganese(III) acetylacetonate, manganese(III) propionate, manganese(III) butyrate, manganese(III) cyclohexane butyrate, and manganese(III) tartrate. In certain embodiments, the manganese(III) reagent is Mn(OAc)₃. In certain embodiments, the manganese(III) reagent is anhydrous Mn(OAc)₃. In some embodiments, the amount of manganese(III) reagent used in step (b) above (or Step (1) of Scheme A) is about 0.5 molar equivalents to about 2 molar equivalents (e.g., about 0.8, 0.9, 1.0, 1.1, 1.2, 1.3, 1.4, or 1.5 molar equivalents) to the nucleoside or analogue thereof of formula I-a or salt thereof. In certain embodiments, about 1 molar equivalent of a manganese(III) reagent (e.g., Mn(OAc)₃) is used. In some embodiments, the manganese(III) reagent and amount used is as depicted in the Examples section.

According to another embodiment, the oxidizing reagent used in step (b) above (or Step (1) of Scheme A) is selected from a mixture of elemental iodine and hydrogen peroxide, hypervalent iodine reagents (e.g., (diacetoxyiodo)benzene, bis(trifluoroacetate)iodobenzene, Togni's reagent, etc.), urea hydrogen peroxide complex, tert-butyl hydroperoxide, silver nitrate/silver sulfate, sodium bromate, ammonium peroxydisulfate, tetrabutylammonium peroxydisulfate, potassium persulfate, Oxone®, Chloramine T, Selectfluor®, Selectfluor® II, sodium hypochlorite, potassium iodate/sodium periodate, N-iodosuccinimide, N-bromosuccinimide, N-chlorosuccinimide, 1,3-diiodo-5,5-dimethylhydantion, pyridinium tribromide, and iodine monochloride or complexes thereof. In certain embodiments, the oxidizing agent is (diacetoxyiodo)benzene (DIB). In some embodiments, the amount of oxidizing reagent used in step (b) above (or Step (1) of Scheme A) is about 1 molar equivalent to about 3 molar equivalents (e.g., about 1.0, 1.1, 1.2, 1.3, 1.4, 1.5, 1.6, 1.7, 1.8, 1.9, or 2.0 molar equivalents) to the nucleoside or analogue thereof of formula I-a or salt thereof. In certain embodiments, about 1.5 molar equivalent of oxidizing reagent (e.g., DIB) is used. In some embodiments, the oxidizing reagent and amount used is as depicted in the Examples section.

According to another embodiment, the conditions used in step (b) above may also include an acid. In some embodiments, the acid is an inorganic acid (e.g., hydrochloric acid, phosphoric acid, sulfuric acid, etc.) or an organic acid (e.g., acetic acid, trifluoroacetetic acid, methansulfonic acid, para-toluenesulfonic acid, etc.). In certain embodiments, the acid is acetic acid (AcOH). In some embodiments, the amount of acid used in step (b) above is about 0.5 molar equivalents to about 2 molar equivalents (e.g., about 0.8, 0.9, 1.0, 1.1, 1.2, 1.3, 1.4, or 1.5 molar equivalents) to the nucleoside or analogue thereof of formula I-a or salt thereof. In certain embodiments, about 1 molar equivalent of acid (e.g., AcOH) is used. In some embodiments, the acid and amount used is as depicted in the Examples section.

According to another embodiment, the conditions used in step (b) above may also include an acetate source. In some embodiments, the acetate source is any organic or inorganic compound that may provide an acetate ion (e.g., AcO⁻) for reaction (e.g., acetic acid, sodium acetate, metal acetates, etc.). In certain embodiments, the acetate source is acetic acid (AcOH). In some embodiments, the amount of acetate source used in step (b) above is about 0.5 molar equivalents to about 2 molar equivalents (e.g., about 0.8, 0.9, 1.0, 1.1, 1.2, 1.3, 1.4, or 1.5 molar equivalents) to the nucleoside or analogue thereof of formula I-a or salt thereof. In certain embodiments, about 1 molar equivalent of acetate source (e.g., AcOH) is used. In some embodiments, the acetate source and amount used is as depicted in the Examples section.

According to another embodiment, the conditions used in step (b) above may also include a solvent. In some embodiments, the solvent is selected from water, acetonitrile, alcohols (e.g., methanol, ethanol, isopropanol, etc.), ethers (diethyl ether, tetrahydrofuran, 2-methyltetrahydrofuran, dioxane, etc.), esters (ethyl acetate, isopropyl acetate, etc.), ketones (e.g., acetone, etc.), halocarbons (dichloromethane, 1,2-dichloroethane, etc.), aromatic hydrocarbons (toluene, xylenes, etc.), N,N-dimethylformamide, N,N-dimethylacetamide, dimethyl sulfoxide, and mixtures thereof. In certain embodiments, the solvent is acetonitrile. In certain embodiments, the solvent is 1,2-dichloroethane (DCE). In some embodiments, the volume (V) of solvent used in step (b) above is about 5 V to about 15 V (e.g., about 6, 7, 8, 9, 10, 11, 12, 13, or 14 volume). In certain embodiments, about 1 V of solvent (e.g., DCE) is used. In some embodiments, the solvent and volume (V) used is as depicted in the Examples section.

According to another embodiment, the conditions used in step (b) above may also include heating the reaction to a temperature for an amount of time. In some embodiments, the heating comprises a temperature of about room temperature (e.g., 20° C.) to about 100° C. (e.g., about 30, 40, 50, 60, 70, 80, or 90° C.) for about 2 hours to about 6 hours (e.g., for about 2, 3, 4, 5, or 6 hours). In some embodiments, the heating comprises a temperature of about room temperature (e.g., 20° C.) to about 100° C. (e.g., about 30, 40, 50, 60, 70, 80, or 90° C.) for about 6 hours to about 48 hours (e.g., for about 8, 10, 12, 14, 16, 18, 20, 22, 24, 26, 28, 30, or 32 hours). In some embodiments, the conditions comprise heating the reaction to about 80° C. for about 24 hours. In some embodiments, the conditions comprise heating the reaction to about 80° C. for about 5 hours. In some embodiments, the reaction temperature and duration used is as depicted in the Examples section.

In some embodiments, a nucleoside (e.g., nucleoside) or analogue thereof of formula I-b is a nucleoside or analogue thereof of formula I-b-1, or I-b-1′, or a mixture thereof:

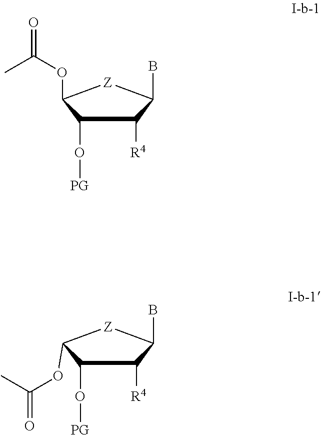

or a salt thereof. In some embodiments, PG on 3′ position is phenyl-C(O)—.

In some embodiments, a nucleoside (e.g., nucleoside) or analogue thereof of formula I-b is a nucleoside or analogue thereof of formula I-b-2, or I-b-2′, or a mixture thereof:

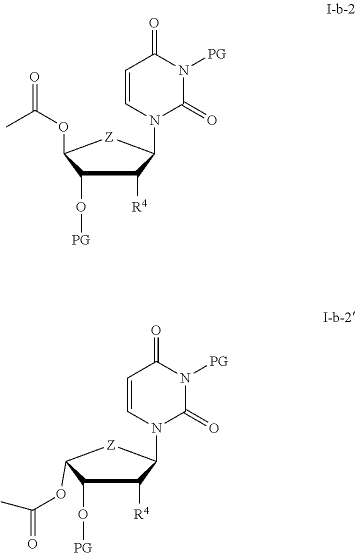

or a salt thereof. In some embodiments, PG on nucleobase is phenyl-CH₂—O—CH₂—, and PG on 3′ position is phenyl-C(O)—. In some embodiments, PG on nucleobase and 3′ position are each phenyl-C(O)—.

In some embodiments, a nucleoside (e.g., nucleoside) or analogue thereof of formula I-b is a nucleoside or analogue thereof of formula I-b-3, or I-b-3′, or a mixture thereof:

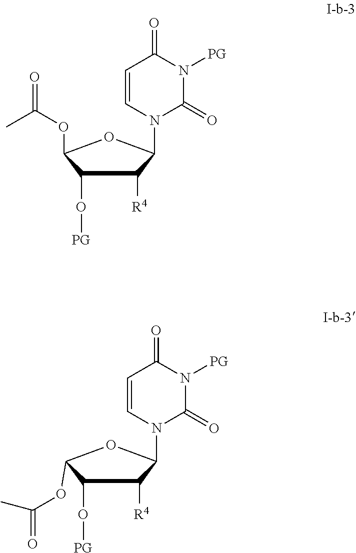

or a salt thereof. In some embodiments, PG on nucleobase is phenyl-CH₂—O—CH₂—, and PG on 3′ position is phenyl-C(O)—. In some embodiments, PG on nucleobase and 3′ position are each phenyl-C(O)—.

In some embodiments, a nucleoside (e.g., nucleoside) or analogue thereof of formula I-b is a nucleoside or analogue thereof of formula I-b-4, or I-b-4′, or a mixture thereof:

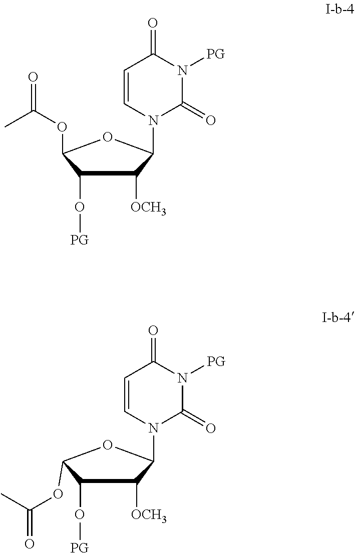

or a salt thereof. In some embodiments, PG on nucleobase is phenyl-CH₂—O—CH₂—, and PG on 3′ position is phenyl-C(O)—. In some embodiments, PG on nucleobase and 3′ position are each phenyl-C(O)—.

In some embodiments, a nucleoside (e.g., nucleoside) or analogue thereof of formula I-b is a nucleoside or analogue thereof of formula I-b-5, or I-b-5′, or a mixture thereof:

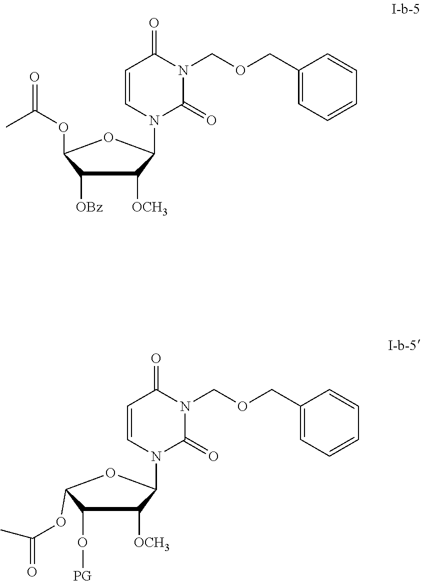

or a salt thereof.

In some embodiments, a nucleoside (e.g., nucleoside) or analogue thereof of formula I-b is a nucleoside or analogue thereof of formula I-b-6, or I-b-6′, or a mixture thereof:

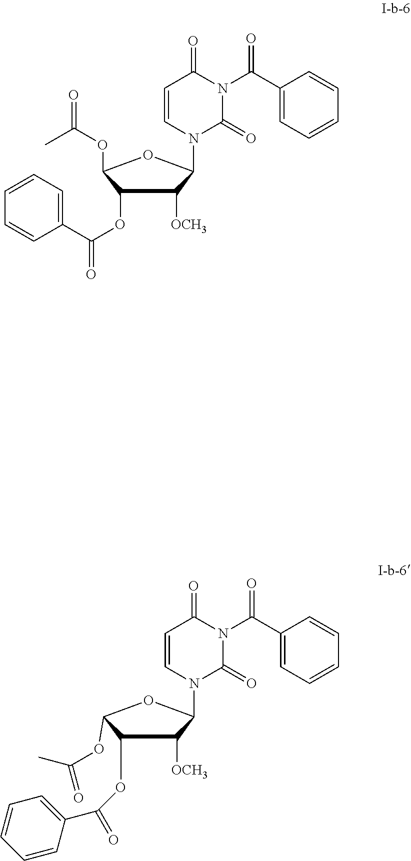

or a salt thereof.

Also provided herein is a process for preparing a nucleoside or analogue thereof comprising a 4′-acetoxy group, wherein the nucleoside or analogue thereof comprising a 4′-acetoxy group is represented by formula I-b-1, or I-b-1′, or a mixture thereof:

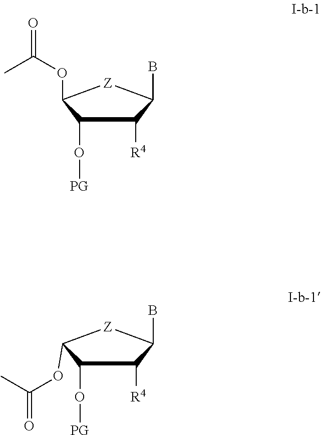

or a salt thereof, comprising the steps:
(a) providing a nucleoside or analogue thereof comprising a 4′-carboxyl group represented by formula I-a-1:

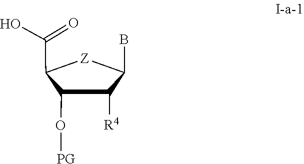

or a salt or ester thereof, and
(b) subjecting the nucleoside or analogue thereof of formula I-a-1 to conditions sufficient to form the nucleoside or analogue thereof of formula I-b-1, or I-b-1′, or a mixture thereof, wherein the conditions sufficient to form the nucleoside or analogue thereof of formula I-b-1, or I-b-1′, or a mixture thereof, comprise the steps:
   (i) combining the nucleoside or analogue thereof of formula I-a-1 with dichloroethane (DCE) to form a mixture;
   (ii) adding AcOH to the mixture of step (i) with agitation;
   (iii) adding Mn(OAc)₂ and (diacetoxyiodo)benzene (DIB) to the agitated mixture of step (ii); and
   (iv) heating the mixture of step (iii) at about 80° C. for about 2 hours to about 24 hours (for example, about 2, 3, 4, 5, 6, 7, 8, 9, 10, 11, 12, 13, 14, 15, 16, 17, 18, 19, 20, 21, 22, 23, or 24 hours), wherein:
each B is independently a nucleobase;
each PG is a protecting group, optionally benzyl or picolyl;
each R⁴ is independently hydrogen, fluoro, —OH, —OC₁₋₆alkyl, —OCH₂CH₂OC₁₋₆alkyl, or —O-protecting group (—OPG);
each Z is independently —O—, —S—, —N(R)—, or —C(R)₂—; and
each R is independently hydrogen, a protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur.

In some embodiments, provided herein is a process for preparing a nucleoside or analogue thereof comprising a 4′-acetoxy group, wherein the nucleoside or analogue thereof comprising a 4′-acetoxy group is represented by formula I-b-2, or I-b-2′, or a mixture thereof:

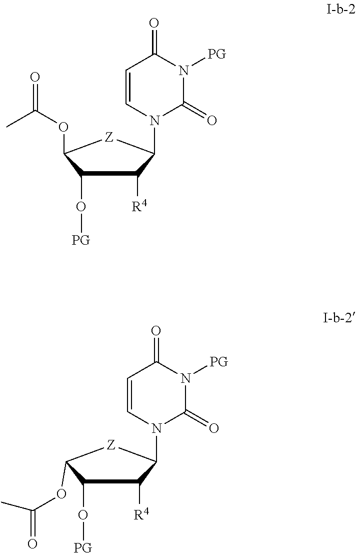

or a salt thereof, comprising the steps:
(a) providing a nucleoside or analogue thereof comprising a 4′-carboxyl group represented by formula I-a-2:

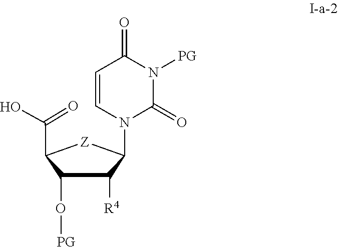

or a salt or ester thereof, and
(b) subjecting the nucleoside or analogue thereof of formula I-a-2 to conditions sufficient to form the nucleoside or analogue thereof of formula I-b-2, or I-b-2′, or a mixture thereof, wherein the conditions sufficient to form the nucleoside or analogue thereof of formula I-b-2, or I-b-2′, or a mixture thereof, comprise the steps:
   (i) combining the nucleoside or analogue thereof of formula I-a-2 with dichloroethane (DCE) to form a mixture;
   (ii) adding AcOH to the mixture of step (i) with agitation;
   (iii) adding Mn(OAc)₂ and (diacetoxyiodo)benzene (DIB) to the agitated mixture of step (ii); and
   (iv) heating the mixture of step (iii) at about 80° C. for about 2 hours to about 24 hours (for example, about 2, 3, 4, 5, 6, 7, 8, 9, 10, 11, 12, 13, 14, 15, 16, 17, 18, 19, 20, 21, 22, 23, or 24 hours), wherein:
PG attaching to the nucleobase is a protecting group, optionally

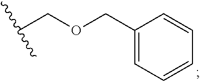 [SMILES: *COCc1ccccc1]

PG attaching the 3′-Oxygen is a protecting group, optionally benzyl or picolyl;
each R⁴ is independently hydrogen, fluoro, —OH, —OC₁₋₆alkyl, —OCH₂CH₂OC₁₋₆alkyl, or —O-protecting group (—OPG);
each Z is independently —O—, —S—, —N(R)—, or —C(R)₂—; and
each R is independently hydrogen, a protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur.

In some embodiments, provided herein is a process for preparing a nucleoside or analogue thereof comprising a 4′-acetoxy group, wherein the nucleoside or analogue thereof comprising a 4′-acetoxy group is represented by formula I-b-3, or I-b-3′, or a mixture thereof:

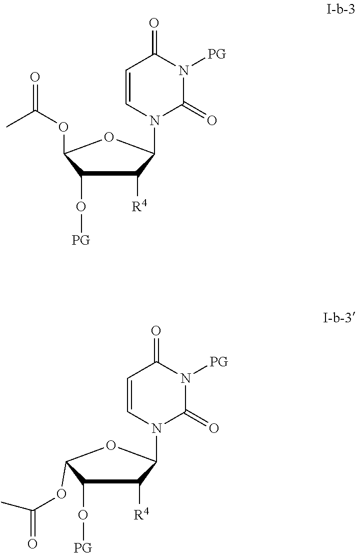

or a salt thereof, comprising the steps:
(a) providing a nucleoside or analogue thereof comprising a 4′-carboxyl group represented by formula I-a-3:

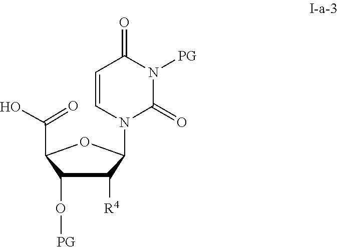

or a salt or ester thereof, and
(b) subjecting the nucleoside or analogue thereof of formula I-a-3 to conditions sufficient to form the nucleoside or analogue thereof of formula I-b-3, or I-b-3′, or a mixture thereof, wherein the conditions sufficient to form the nucleoside or analogue thereof of formula I-b-3, or I-b-3′, or a mixture thereof, comprise the steps:
   (i) combining the nucleoside or analogue thereof of formula I-a-3 with dichloroethane (DCE) to form a mixture;
   (ii) adding AcOH to the mixture of step (i) with agitation;
   (iii) adding Mn(OAc)₂ and (diacetoxyiodo)benzene (DIB) to the agitated mixture of step (ii); and
   (iv) heating the mixture of step (iii) at about 80° C. for about 2 hours to about 24 hours (for example, about 2, 3, 4, 5, 6, 7, 8, 9, 10, 11, 12, 13, 14, 15, 16, 17, 18, 19, 20, 21, 22, 23, or 24 hours), wherein:
PG attaching to the nucleobase is a protecting group, optionally

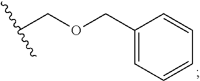 [SMILES: *COCc1ccccc1]

PG attaching the 3′-Oxygen is a protecting group, optionally benzyl or picolyl; and
each R⁴ is independently hydrogen, fluoro, —OH, —OC₁₋₆alkyl, —OCH₂CH₂OC₁₋₆alkyl, or —O-protecting group (—OPG).

In some embodiments, provided herein is a process for preparing a nucleoside or analogue thereof comprising a 4′-acetoxy group, wherein the nucleoside or analogue thereof comprising a 4′-acetoxy group is represented by formula I-b-4, or I-b-4′, or a mixture thereof:

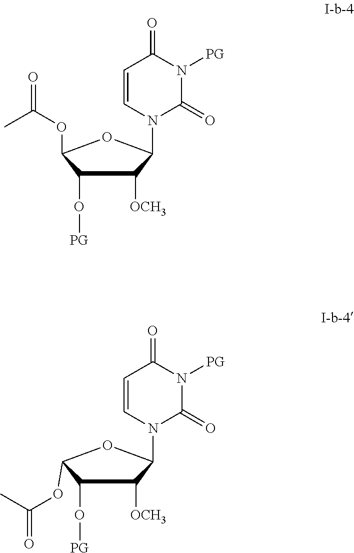

or a salt thereof, comprising the steps:
(a) providing a nucleoside or analogue thereof comprising a 4′-carboxyl group represented by formula I-a-4:

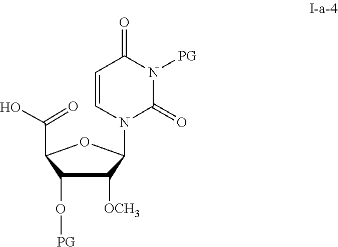

or a salt or ester thereof, and
(b) subjecting the nucleoside or analogue thereof of formula I-a-4 to conditions sufficient to form the nucleoside or analogue thereof of formula I-b-4, or I-b-4′, or a mixture thereof, wherein the conditions sufficient to form the nucleoside or analogue thereof of formula I-b-4, or I-b-4′, or a mixture thereof, comprise the steps:
   (i) combining the nucleoside or analogue thereof of formula I-a-4 with dichloroethane (DCE) to form a mixture;
   (ii) adding AcOH to the mixture of step (i) with agitation;
   (iii) adding Mn(OAc)₂ and (diacetoxyiodo)benzene (DIB) to the agitated mixture of step (ii); and
   (iv) heating the mixture of step (iii) at about 80° C. for about 2 hours to about 24 hours (for example, about 2, 3, 4, 5, 6, 7, 8, 9, 10, 11, 12, 13, 14, 15, 16, 17, 18, 19, 20, 21, 22, 23, or 24 hours), wherein:
PG attaching to the nucleobase is a protecting group, optionally

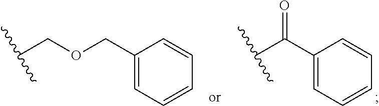 [SMILES: *COCc1ccccc1 ; *C(=O)c1ccccc1]

and
PG attaching the 3′-Oxygen is a protecting group, optionally benzyl or picolyl.

In some embodiments, provided herein is a process for preparing a nucleoside or analogue thereof comprising a 4′-acetoxy group, wherein the nucleoside or analogue thereof comprising a 4′-acetoxy group is represented by formula I-b-5, or I-b-5′, or a mixture thereof:

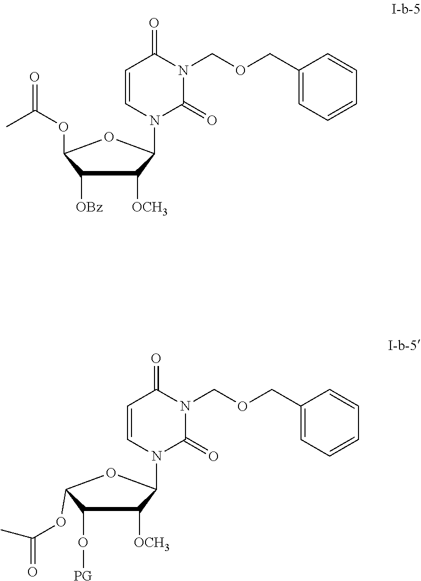

or a salt thereof, comprising the steps:
(a) providing a nucleoside or analogue thereof comprising a 4′-carboxyl group represented by formula I-a-5:

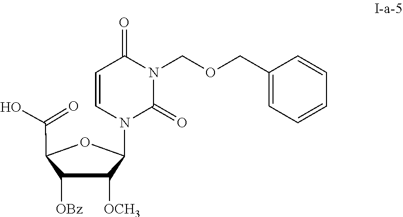

or a salt or ester thereof, and
(b) subjecting the nucleoside or analogue thereof of formula I-a-5 to conditions sufficient to form the nucleoside or analogue thereof of formula I-b-5, or I-b-5′, or a mixture thereof, wherein the conditions sufficient to form the nucleoside or analogue thereof of formula I-b-5, or I-b-5′, or a mixture thereof, comprise the steps:
   (i) combining the nucleoside or analogue thereof of formula I-a-5 with dichloroethane (DCE) to form a mixture;
   (ii) adding AcOH to the mixture of step (i) with agitation;
   (iii) adding Mn(OAc)₂ and (diacetoxyiodo)benzene (DIB) to the agitated mixture of step (ii); and
   (iv) heating the mixture of step (iii) at about 80° C. for about 2 hours to about 24 hours (for example, about 2, 3, 4, 5, 6, 7, 8, 9, 10, 11, 12, 13, 14, 15, 16, 17, 18, 19, 20, 21, 22, 23, or 24 hours).

In some embodiments, provided herein is a process for preparing a nucleoside or analogue thereof comprising a 4′-acetoxy group, wherein the nucleoside or analogue thereof comprising a 4′-acetoxy group is represented by formula I-b-6, or I-b-6′, or a mixture thereof:

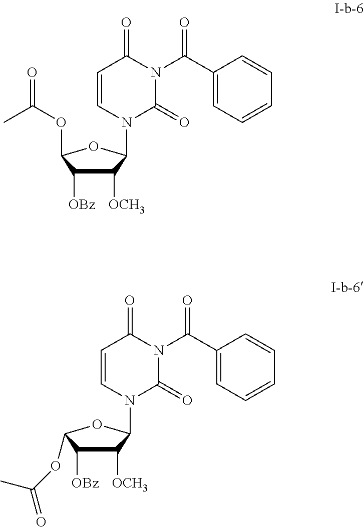

or a salt thereof, comprising the steps:
(a) providing a nucleoside or analogue thereof comprising a 4′-carboxyl group represented by formula I-a-6:

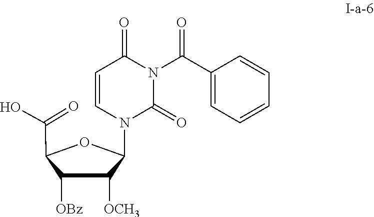

or a salt or ester thereof, and
(b) subjecting the nucleoside or analogue thereof of formula I-a-6 to conditions sufficient to form the nucleoside or analogue thereof of formula I-b-6, or I-b-6′, or a mixture thereof, wherein the conditions sufficient to form the nucleoside or analogue thereof of formula I-b-6, or I-b-6′, or a mixture thereof, comprise the steps:
   (i) combining the nucleoside or analogue thereof of formula I-a-6 with dichloroethane (DCE) to form a mixture;
   (ii) adding AcOH to the mixture of step (i) with agitation;
   (iii) adding Mn(OAc)₂ and (diacetoxyiodo)benzene (DIB) to the agitated mixture of step (ii); and
   (iv) heating the mixture of step (iii) at about 80° C. for about 2 hours to about 24 hours (for example, about 2, 3, 4, 5, 6, 7, 8, 9, 10, 11, 12, 13, 14, 15, 16, 17, 18, 19, 20, 21, 22, 23, or 24 hours).

In some embodiments, provided herein is a process for preparing a nucleoside or analogue thereof comprising a 4′-acetoxy group, wherein the nucleoside or analogue thereof comprising a 4′-acetoxy group is represented by formula I-b-6, or I-b-6′, or a mixture thereof:

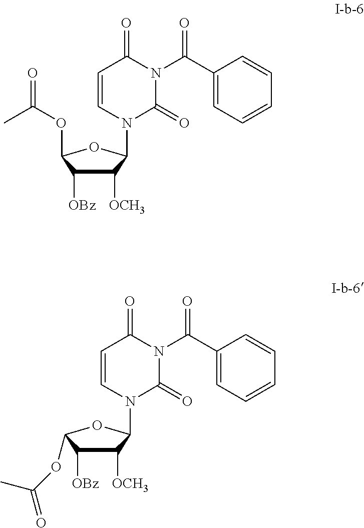

or a salt thereof, comprising the steps:
(a) providing a nucleoside or analogue thereof comprising a 4′-carboxyl group represented by formula I-a-6:

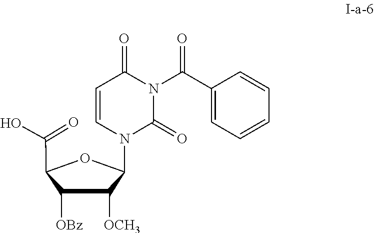

or a salt or ester thereof, and
(b) subjecting the nucleoside or analogue thereof of formula I-a-6 to conditions sufficient to form the nucleoside or analogue thereof of formula I-b-6, or I-b-6′, or a mixture thereof, wherein the conditions sufficient to form the nucleoside or analogue thereof of formula I-b-6, or I-b-6′, or a mixture thereof, comprise the steps:
   (i) combining the nucleoside or analogue thereof of formula I-a-6 with dichloroethane (DCE) to form a mixture;
   (ii) adding AcOH to the mixture of step (i) with agitation;
   (iii) adding Mn(OAc)₃ to the agitated mixture of step (ii); and
   (iv) heating the mixture of step (iii) at about 80° C. for about 2 hours to about 24 hours (for example, about 2, 3, 4, 5, 6, 7, 8, 9, 10, 11, 12, 13, 14, 15, 16, 17, 18, 19, 20, 21, 22, 23, or 24 hours).

In some embodiments, a nucleoside (e.g., nucleoside) or analogue thereof of formula I-a is a nucleoside or analogue thereof of formula I-a-1:

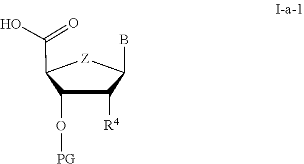

or a salt thereof.

In some embodiments, a nucleoside (e.g., nucleoside) or analogue thereof of formula I-a is a nucleoside or analogue thereof of formula I-a-2:

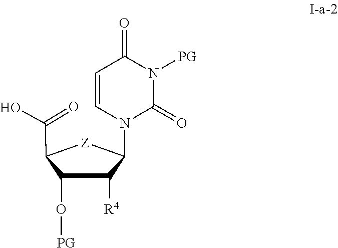

or a salt thereof.

In some embodiments, a nucleoside (e.g., nucleoside) or analogue thereof of formula I-a is a nucleoside or analogue thereof of formula I-a-3:

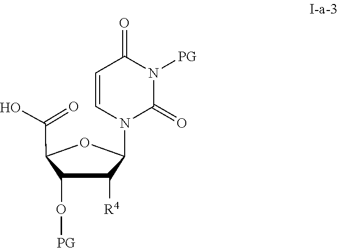

or a salt thereof.

In some embodiments, a nucleoside (e.g., nucleoside) or analogue thereof of formula I-a is a nucleoside or analogue thereof of formula I-a-4:

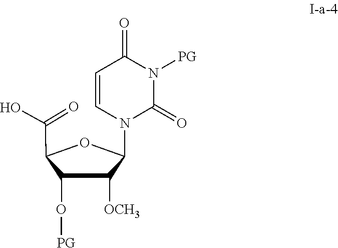

or a salt thereof.

In some embodiments, a nucleoside (e.g., nucleoside) or analogue thereof of formula I-a is a nucleoside or analogue thereof of formula I-a-5:

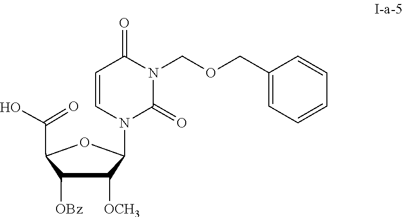

or a salt thereof.

In some embodiments, a nucleoside (e.g., nucleoside) or analogue thereof of formula I-a is a nucleoside or analogue thereof of formula I-a-6:

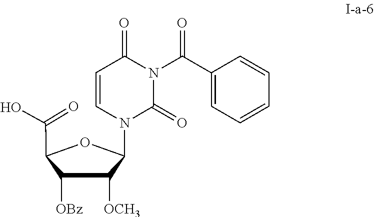

or a salt thereof.

According to another aspect, the present invention provides a process for preparing a nucleotide or analogue of formula I-d:

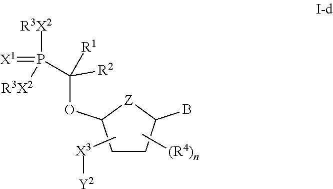

or a salt thereof, comprising the steps:
(a) providing a nucleoside or analogue thereof of formula I-b:

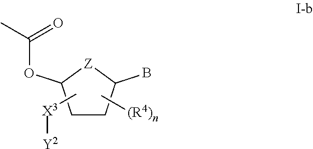

or a salt thereof, and
(b) reacting the nucleoside or analogue thereof of formula I-b with a compound of formula I-C:

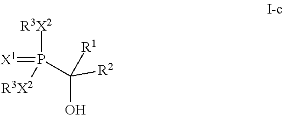

to form the nucleotide or analogue thereof of formula I-d, wherein:
each B is a nucleobase or hydrogen;
R¹ and R² are independently hydrogen or C₁₋₆alkyl;
each R³ is independently hydrogen, a protecting group (PG), a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, fluoro, —OH, —OC₁₋₆alkyl, —OCH₂CH₂OC₁₋₆alkyl, or —O-protecting group (—OPG);
X¹ is O, S, or NR;
each R is independently hydrogen, a protecting group (PG), or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur;
each X² is independently —O—, —S—, —B(H)₂—, or a covalent bond;
X³ is —O—, —S—, or —N(R)—;
Y² is hydrogen or a protecting group (PG);
each Z is independently —O—, —S—, —N(R)—, or —C(R)₂—; and
each n is independently 0, 1, 2, 3, 4, or 5.

According to one embodiment, a nucleoside or analogue thereof of formula I-b is reacted with a nucleoside or analogue thereof of formula I-c in step (b) above in the presence of a Lewis acid to afford a nucleotide, or analogue thereof of formula I-d. Suitable Lewis acids include those that are well known in the art, such as boron trifluoride etherate, thioetherates, and alcohol complexes, dicyclohexylboron triflate, trimethylsilyl triflate, tetrafluoroboric acid, aluminum isopropoxide, silver triflate, silver tetrafluoroborate, titanium trichloride, tin tetrachloride, scandium triflate, copper (II) triflate, zinc iodide, zinc bromide, zinc chloride, ferric bromide, and ferric chloride, or a montmorillonite clay. Suitable Lewis acids may also include BrØnsted acids, such as hydrochloric acid, toluenesulfonic acid, trifluoroacetic acid, or acetic acid. In certain embodiments, a nucleoside or analogue thereof of formula I-b is reacted with a compound of formula I-c in the presence of boron trifluoride etherate or trimethylsilyl triflate to afford a nucleotide or analogue thereof of formula I-d. In some embodiments, a compound of formula I-c is dimethyl hydroxymethyl phosphonate. In some embodiments, a nucleoside or analogue thereof of formula I-b is reacted with a compound of formula I-c to afford a nucleotide or analogue thereof of formula I-d in the presence of a solvent, wherein the solvent can be any solvent described or disclosed herein. In some embodiments, the solvent is an ether (e.g., tetrahydrofuran) or halocarbon (e.g., dichloromethane). In some embodiments, the conditions used to form the nucleotide or analogue thereof of formula I-d from the reaction of a nucleoside or analogue thereof of formula I-b with a compound of formula I-c is as depicted in the Examples section.

In some embodiments, the nucleoside, nucleotide, or analogue thereof of formula I-d is a nucleic acid or analogue thereof of formula I-d-1:

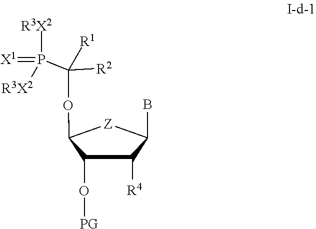

or a salt thereof. In some embodiments, PG on 3′ position is benzoyl.

In some embodiments, the nucleoside, nucleotide, or analogue thereof of formula I-d is a nucleoside, nucleotide, or analogue thereof of formula I-d-2:

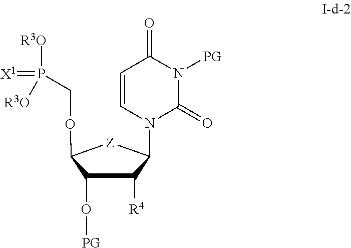

or a salt thereof. In some embodiments, PG on nucleobase is phenyl-CH₂—O—CH₂—, and PG on 3′ position is benzoyl. In some embodiments, PG on nucleobase and 3′ position are each benzoyl.

In some embodiments, the nucleoside, nucleotide, or analogue thereof of formula I-d is a nucleoside, nucleotide, or analogue thereof of formula I-d-3:

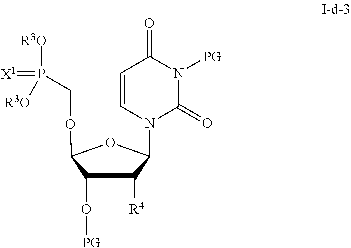

or a salt thereof. In some embodiments, PG on nucleobase is phenyl-CH₂—O—CH₂—, and PG on 3′ position is benzoyl. In some embodiments, PG on nucleobase and 3′ position are each benzoyl.

In some embodiments, the nucleoside, nucleotide, or analogue thereof of formula I-d is a nucleoside, nucleotide, or analogue thereof of formula I-d-4:

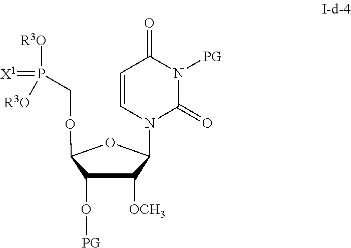

or a salt thereof. In some embodiments, PG on nucleobase is phenyl-CH₂—O—CH₂—, and PG on 3′ position is benzoyl. In some embodiments, PG on nucleobase and 3′ position are each benzoyl.

In some embodiments, the nucleoside, nucleotide, or analogue thereof of formula I-d is a nucleoside, nucleotide, or analogue thereof of formula I-d-5:

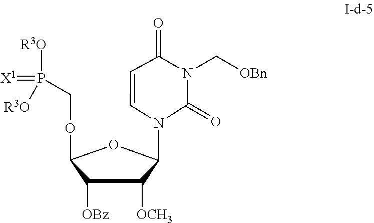

or a salt thereof.

In some embodiments, the nucleoside, nucleotide, or analogue thereof of formula I-d is a nucleoside, nucleotide, or analogue thereof of formula I-d-6:

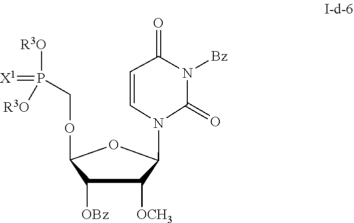

or a salt thereof.

In some embodiments, the nucleoside, nucleotide, or analogue thereof of formula I-d is a nucleoside, nucleotide, or analogue thereof of formula I-d-7:

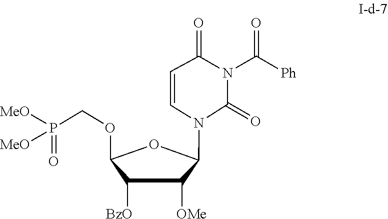

or a salt thereof.

In some embodiments, the nucleoside, nucleotide, or analogue thereof of formula I-d is a nucleoside, nucleotide, or analogue thereof of formula I-d-8:

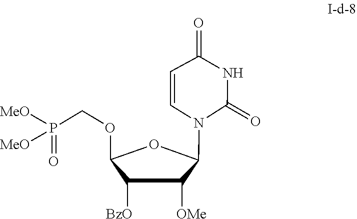

or a salt thereof.

In some embodiments, provided herein is a process for preparing a nucleoside or analogue thereof of formula I-d-7 or I-d-8:

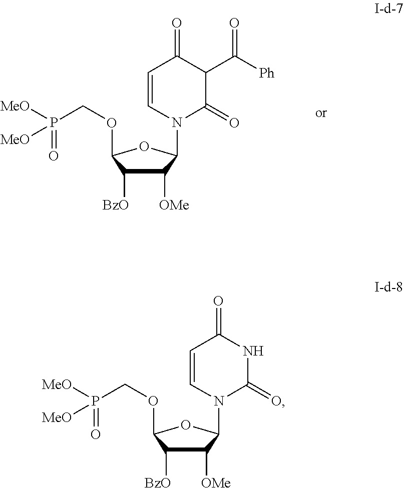

or a salt thereof, or a mixture thereof, comprising the steps:
(a) providing a nucleoside or analogue thereof of formula:

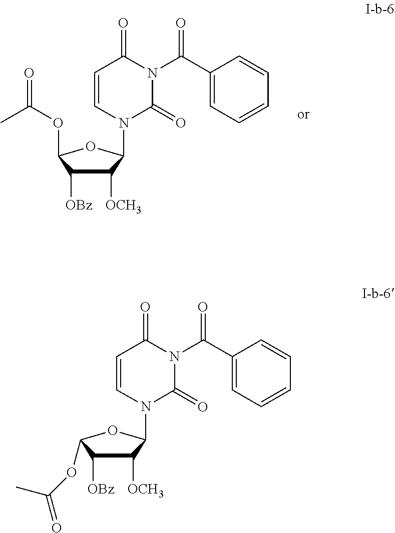

or a salt thereof, or a mixture thereof,
and
(b) reacting the nucleoside or analogue thereof of formula I-b-6, or I-b-6′, or a salt thereof, or a mixture thereof, with a compound of formula

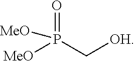

In some embodiments, the reaction conditions are selected from those described in the examples, for example, in Example 6.

According to another aspect, the present invention provides a process for preparing a nucleoside, nucleotide, or analogue of formula I-e:

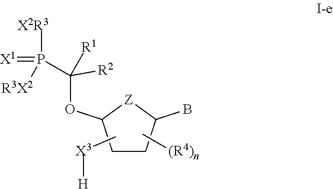

or a salt thereof, comprising the steps:
(a) providing a nucleoside, nucleotide, or analogue thereof of formula I-d:

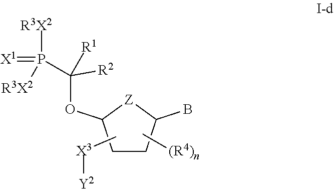

or a salt thereof, and
(b) deprotecting the nucleoside, nucleotide, or analogue thereof of formula I-d to form the nucleoside, nucleotide, or analogue thereof of formula I-e, wherein:
each B is a nucleobase or hydrogen;
R¹ and R² are independently hydrogen or C₁₋₆alkyl;
each R³ is independently hydrogen, a protecting group (PG), a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, fluoro, —OH, —OC₁₋₆alkyl, —OCH₂CH₂OC₁₋₆alkyl, or —O-protecting group (—OPG);
X¹ is O, S, or NR;
each R is independently hydrogen, a protecting group (PG), or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur;
each X² is independently —O—, —S—, —B(H)₂—, or a covalent bond;
X³ is —O—, —S—, or —N(R)—;
Y² is a protecting group (PG);
each Z is independently —O—, —S—, —N(R)—, or —C(R)₂—; and
each n is independently 0, 1, 2, 3, 4, or 5.

According to embodiments described herein, the deprotection of a protecting group (PG) in step (b) above includes those protecting groups described in detail in Protecting Groups in Organic Synthesis, (T. W. Greene and P. G. M. Wuts, 3^{rd} edition, John Wiley & Sons, 1999), the entirety of each of which is herein incorporated by reference. In some embodiments, the protecting group is a suitable hydroxyl protecting group, a suitable amino protection group, or a suitable thiol protecting group. In certain embodiments, the hydroxyl protecting group is benzyl or picolyl.

As used herein, the phrase “suitable hydroxyl protecting group” are well known in the art and when taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, picolinate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl, 3,4-dimethoxybenzyl, o-nitrobenzyl, p-nitrobenzyl, p-halobenzyl, 2,6-dichlorobenzyl, p-cyanobenzyl, and 2- and 4-picolyl. In some embodiments, the suitable hydroxyl protecting group is an acid labile group such as trityl, 4-methyoxytrityl, 4,4′-dimethyoxytrityl (DMTr), 4,4′,4″-trimethyoxytrityl, 9-phenyl-xanthen-9-yl, 9-(p-tolyl)-xanthen-9-yl, pixyl, 2,7-dimethylpixyl, and the like, suitable for deprotection during both solution-phase and solid-phase synthesis of acid-sensitive oligonucleotides using for example, dichloroacetic acid, trichloroacetic acid, trifluoroacetic acid, or acetic acid. The t-butyldimethylsilyl group is stable under the acidic conditions used to remove the DMTr group during synthesis but can be removed after cleavage and deprotection of the RNA oligomer with a fluoride source, e.g., tetrabutylammonium fluoride or pyridine hydrofluoride.

As used herein, the phrase “suitable amino protecting group” are well known in the art and when taken with the nitrogen to which it is attached, include, but are not limited to, aralkylamines, carbamates, allyl amines, amides, and the like. Examples of mono-protection groups for amines include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like. Examples of di-protection groups for amines include amines that are substituted with two substituents independently selected from those described above as mono-protection groups, and further include cyclic imides, such as phthalimide, maleimide, succinimide, 2,2,5,5-tetramethyl-1,2,5-azadisilolidine, azide, and the like. It will be appreciated that upon acid hydrolysis of an amino protecting groups, a salt compound thereof is formed. For example, when an amino protecting group is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms are contemplated.

As used herein, the phrase “suitable thiol protecting group” further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

According to embodiments described herein, the deprotecting of a nucleoside, nucleotide, or analogue thereof of formula I-d to form the nucleoside, nucleotide or analogue thereof of formula I-e in step (b) above can include the deprotection of any suitable protection group disclosed above or defined herein. In certain embodiments, a nucleoside, nucleotide, or analogue thereof of formula I-d comprises a 4′-O-methylene phosphonate ester and mono-deprotection is performed under basic aqueous conditions. Suitable bases include metal hydroxides (e.g., sodium hydroxide, potassium hydroxide, lithium hydroxide and barium hydroxide), metal carbonates (e.g., lithium carbonate, sodium carbonate, potassium carbonate, calcium carbonate, cesium carbonate), sodium hydrogen carbonate, organic amines (e.g., triethylamine, N,N-diisopropylethylamine (DIEA), N-methylmorpholine, N-ethylmorpholine, tributylamine, 1,4-diazabicyclo[2.2.2]octane (DABCO), N-methylimidazole (NMI), pyridine, 2,6-lutidine, 2,4,6-collidine, 4-dimethylaminopyridine (DMAP), 1,8-bis(dimethylamino)naphthalene (“proton sponge”), 1,8-diazabicyclo[5.4.0]undec-7-ene (DBU), 1,5-diazabicyclo[4.3.0]non-5-ene (DBN), 7-methyl-1,5,7-triazabicyclo[4.4.0]dec-5-ene (MTBD), 2-tert-butyl-1,1,3,3-tetramethylguanidine, 2,8,9-trimethyl-2,5,8,9-tetraaza-1-phosphabicyclo[3.3.3]undecane or phosphazene base). In certain embodiments, X³ is —O— and the suitable hydroxyl protecting group is an ester protection group (e.g., benzoate or picoloyl ester) which is deprotected using a metal carbonate (e.g., potassium carbonate) in an alcohol solvent (e.g., methanol). In some embodiments, the conditions used for deprotecting of a nucleoside, nucleotide, or analogue thereof of formula I-d to form the nucleoside, nucleotide or analogue thereof of formula I-e is as depicted in the Examples section.

In some embodiments, the nucleoside, nucleotide, or analogue thereof of formula I-e is a nucleoside, nucleotide or analogue thereof of formula I-e-1:

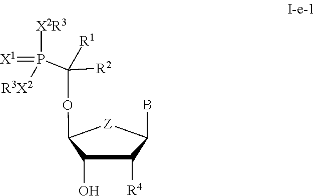

or a salt thereof.

In some embodiments, the nucleoside, nucleotide, or analogue thereof of formula I-e is a nucleoside, nucleotide or analogue thereof of formula I-e-2:

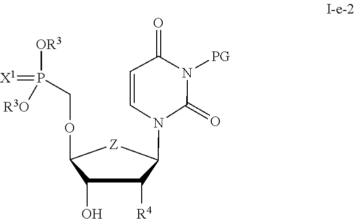

or a salt thereof.

In some embodiments, the nucleoside, nucleotide, or analogue thereof of formula I-e is a nucleoside, nucleotide or analogue thereof of formula I-e-3:

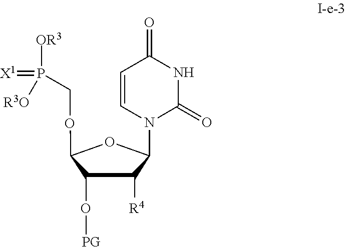

or a salt thereof. In some embodiments, PG on 3′ position is benzoyl.

In some embodiments, the nucleoside, nucleotide, or analogue thereof of formula I-e is a nucleoside, nucleotide or analogue thereof of formula I-e-4:

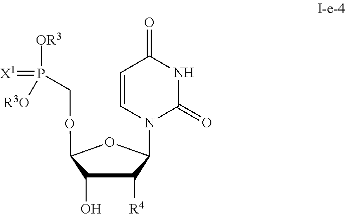

or a salt thereof.

In some embodiments, the nucleoside, nucleotide, or analogue thereof of formula I-e is a nucleoside, nucleotide or analogue thereof of formula I-e-5:

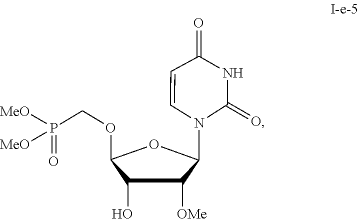

or a salt thereof.

In some embodiments, provided herein is a process for preparing a nucleoside or analogue thereof of formula:

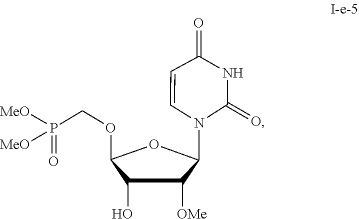

or a salt thereof, comprising the steps:
(a) providing a nucleoside or analogue thereof of formula:

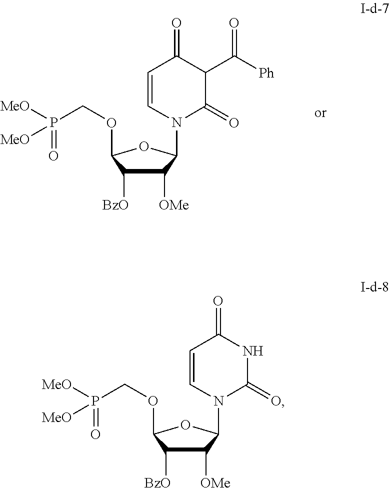

or a salt thereof, or a mixture thereof,
and
(b) deprotecting the nucleoside or analogue thereof of formula I-d-7, or I-d-8, or a salt thereof, or a mixture thereof, to form the nucleoside or analogue thereof of formula I-e-5. In some embodiments, the deprotecting conditions are selected from those described in the examples, for example, in Example 6.

According to another aspect, the present invention provides a process for preparing a nucleoside, nucleotide, or analogue of formula I-g:

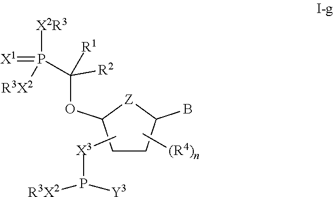

or a salt thereof, comprising the steps:
(a) providing a nucleoside, nucleotide, or analogue thereof of formula I-e:

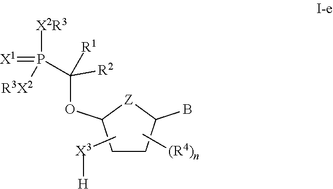

or a salt thereof, and
(b) reacting the nucleoside, nucleotide, or analogue thereof of formula I-e with a compound of formula I-f:

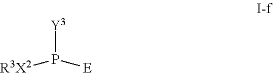

to form the nucleotide or analogue thereof of formula I-g, wherein:
each B is a nucleobase or hydrogen;
E is halogen or —NR₂;
R¹ and R² are independently hydrogen or C₁₋₆alkyl;
each R³ is independently hydrogen, a protecting group (PG), a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, fluoro, —OH, —OC₁₋₆alkyl, —OCH₂CH₂OC₁₋₆alkyl, or —O— protecting group (—OPG);
X¹ is O, S, or NR;
each R is independently hydrogen, a protecting group (PG), or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur;
each X² is independently —O—, —S—, —B(H)₂—, or a covalent bond;
X³ is —O—, —S—, or —N(R)—;
Y³ is halogen or —NR₂;
each Z is independently —O—, —S—, —N(R)—, or —C(R)₂—; and
each n is independently 0, 1, 2, 3, 4, or 5.

In certain embodiments, I-f is represented by formula I-f-1:

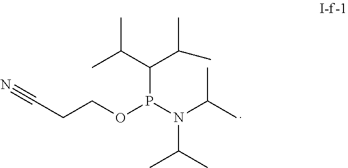 [SMILES: CC(C)C(C(C)C)P(OCCC#N)N(C(C)C)C(C)C]

In certain embodiments, I-f is represented by formula I-f-2:

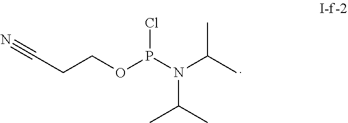

According to one embodiment, a compound of formula I-f, I-f-1, or I-f-2 in step (b) above is a P(III) forming reagent commonly used to prepare phosphoramidites or analogues thereof in oligonucleotide syntheses. In some embodiments, the P(III) forming reagent is 2-cyanoethyl N,N-diisopropylchlorophosphoramidite, in the presence of a base Suitable bases used in the reaction are well known in the art and include organic and inorganic bases. In some embodiments, the base is a tertiary amine such as triethylamine or diisopropylethylamine. In certain embodiments, the base is 1-methylimidazole (NMI). In certain embodiments 2-Cyanoethyl N,N,N′,N′-tetraisopropylphosphorodiamidite is used in the presence of a weak acid catalyst. In certain embodiments, the weak acid catalyst is tetrazole or 4,5-dicyanoimidazole.

In some embodiments, the solvent is a commonly used organic solvent. In certain additional embodiments, the solvents is dichloromethane (DCM), acetonitrile (ACN), or tetrahydrofuran (THF). In certain embodiments, the solvent is an ether (e.g., tetrahydrofuran), a nitrile (acetonitrile) or a halocarbon (e.g., dichloromethane).

In some embodiments, the conditions used to form the nucleoside, nucleotide, or analogue thereof of formula I-g by reacting a nucleoside, nucleotide or analogue thereof of formula I-e with a compound of formula I-f is as depicted in the Examples section.

In some embodiments, the nucleoside, nucleotide, or analogue thereof of formula I-g is a nucleoside, nucleotide or analogue thereof of formula I-g-1:

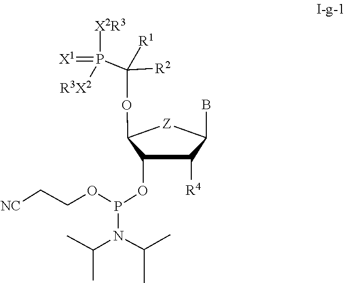

or a salt thereof.

As defined above and described herein, each B is independently a nucleobase or hydrogen.

In some embodiments, B is a nucleobase. In some embodiments, B is hydrogen.

In some embodiments, B is a protected nucleobase (e.g., a nucleobase containing a PG group). In some embodiments, B is

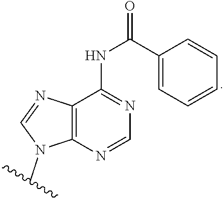 [SMILES: *n1cnc2c(NC(=O)c3ccccc3)ncnc21]

In some embodiments, B is

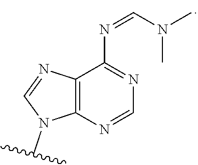 [SMILES: *n1cnc2c(/N=C\N(C)C)ncnc21]

In some embodiments, B is

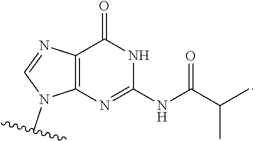 [SMILES: *n1cnc2c(=O)[nH]c(NC(=O)C(C)C)nc21]

In some embodiments, B is

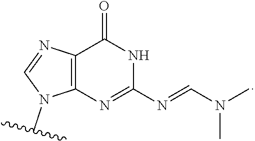 [SMILES: *n1cnc2c(=O)[nH]c(/N=C/N(C)C)nc21]

In some embodiments, B is

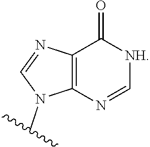 [SMILES: *n1cnc2c(=O)[nH]cnc21]

In some embodiments, B is

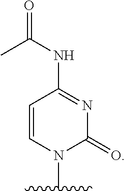 [SMILES: *n1ccc(NC(C)=O)nc1=O]

In some embodiments, B is

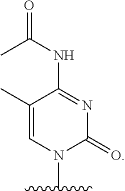 [SMILES: *n1cc(C)c(NC(C)=O)nc1=O]

In some embodiments, B is

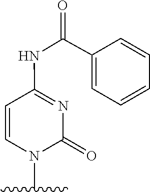 [SMILES: *n1ccc(NC(=O)c2ccccc2)nc1=O]

In some embodiments, B is

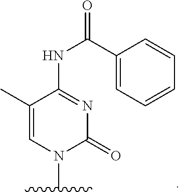 [SMILES: *n1cc(C)c(NC(=O)c2ccccc2)nc1=O]

In some embodiments, B is

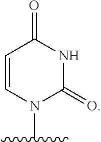 [SMILES: *n1ccc(=O)[nH]c1=O]

In some embodiments, B is

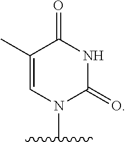 [SMILES: *n1cc(C)c(=O)[nH]c1=O]

In some embodiments, B is

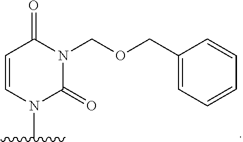 [SMILES: *n1ccc(=O)n(COCc2ccccc2)c1=O]

In some embodiments, B is

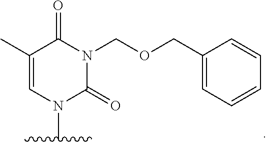 [SMILES: *n1cc(C)c(=O)n(COCc2ccccc2)c1=O]

In some embodiments, B is

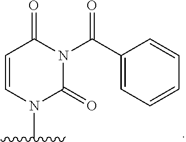 [SMILES: *n1ccc(=O)n(C(=O)c2ccccc2)c1=O]

In some embodiments, B is

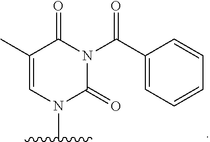 [SMILES: *n1cc(C)c(=O)n(C(=O)c2ccccc2)c1=O]

In some embodiments, B is as depicted in the nucleosides of the Examples section.

As defined above and described herein, R¹ and R² are independently hydrogen or C₁₋₆alkyl.

In some embodiments, R¹ is hydrogen. In some embodiments, R¹ is C₁₋₆alkyl. In some embodiments, R¹ is methyl.

In some embodiments, R² is hydrogen. In some embodiments, R² is C₁₋₆alkyl. In some embodiments, R² is methyl.

In some embodiments, R¹ and R² are as depicted in the nucleosides of the Examples section.

As defined above and described herein, each R³ is independently hydrogen, a protecting group (PG), a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R³ is hydrogen. In some embodiments, R³ is a protecting group (PG). In some embodiments, R³ is a suitable prodrug. In some embodiments, R³ is an optionally substituted C₁₋₆ aliphatic. In some embodiments, R³ is an optionally substituted phenyl. In some embodiments, R³ is an optionally substituted 4-7 membered saturated or partially unsaturated heterocyclic having 1-2 heteroatoms independently selected from nitrogen, oxygen, and sulfur. In some embodiments, R³ is an optionally substituted 5-6 membered heteroaryl ring having 1-4 heteroatoms independently selected from nitrogen, oxygen, and sulfur.

In some embodiments, R³ is methyl. In some embodiments, R³ is —OCH₂CH₂CN.

As defined above and described herein, each R⁴ is independently hydrogen, fluoro, —OH, —OC₁₋₆alkyl, —OCH₂CH₂OC₁₋₆alkyl, or —O-protecting group (—OPG).

In some embodiments, R⁴ is hydrogen. In some embodiments, R⁴ is fluoro. In some embodiments, R⁴ is —OH. In some embodiments, R⁴ is —OC₁₋₆alkyl. In some embodiments, R⁴ is —OMe. In some embodiments, R⁴ is —OCH₂CH₂OC₁₋₆alkyl. In some embodiments, R⁴ is —OCH₂CH₂OMe. In some embodiments, R⁴ is —O-protecting group (—OPG).

In some embodiments, nucleoside, nucleotide, or analogue thereof of formula I-g is a nucleoside or analogue thereof of formula I-g-2:

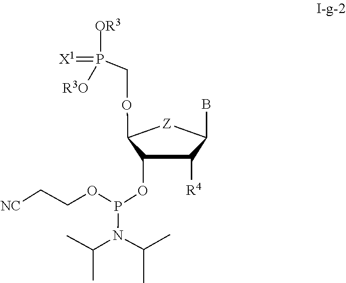

or a salt thereof.

In some embodiments, nucleoside, nucleotide, or analogue thereof of formula I-g is a nucleoside, nucleotide or analogue thereof of formula I-g-3:

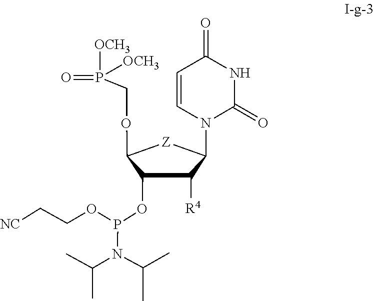

or a salt thereof.

In some embodiments, nucleoside, nucleotide, or analogue thereof of formula I-g is a nucleoside, nucleotide or analogue thereof of formula I-g-4:

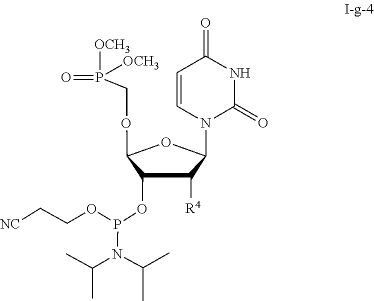

or a salt thereof.

In some embodiments, nucleoside, nucleotide, or analogue thereof of formula I-g is a nucleoside, nucleotide or analogue thereof of formula I-g-5:

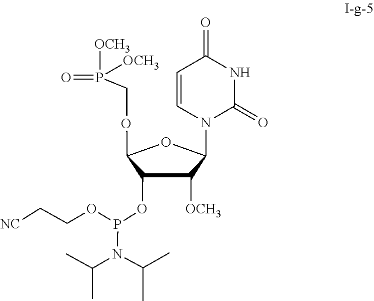

or a salt thereof.

In some embodiments, R⁴ is as depicted in the nucleosides of the Examples section.

As defined above and described herein, each X¹ is independently O, S, or NR.

In some embodiments, X¹ is O. In some embodiments, X¹ is S. In some embodiments, X¹ is NR.

In some embodiments, X¹ is as depicted in the nucleosides of the Examples section.

As defined above and described herein, each X² is independently each X² is independently —O—, —S—, —B(H)₂—, or a covalent bond.

In some embodiments, X² is —O—. In some embodiments, X² is —S—. In some embodiments, X² is —B(H)₂—. In some embodiments, X² is a covalent bond.

In some embodiments, X² is as depicted in the nucleosides of the Examples section.

As defined above and described herein, each X³ is independently —O—, —S—, or —N(R)—.

In some embodiments, X³ is —O—. In some embodiments, X³ is —S—. In some embodiments, X³ is —N(R)—.

In some embodiments, X³ is as depicted in the nucleosides of the Examples section.

As defined above and described herein, each R is independently hydrogen, a protecting group (PG), or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur.

In some embodiments, R is hydrogen. In some embodiments, R is a protecting group (PG). In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom are taken together with their intervening atoms to form an optionally substituted 4-7 membered saturated or partially unsaturated carbocyclic or heterocyclic ring having 0-3 heteroatoms, independently selected from nitrogen, oxygen, and sulfur.

In some embodiments, R is as depicted in the nucleosides of the Examples section.

As defined above and described herein, E is halogen or —NR₂.

In some embodiments, E is a halogen. In some embodiments, E is —NR₂. In some embodiments, E is a chloro. In some embodiments, E is —N(iPr)₂.

In some embodiments, E is as depicted in the nucleosides of the Examples section.

As defined above and described herein, each Y² is independently hydrogen or a protecting group (PG).

In some embodiments, Y² is hydrogen. In some embodiments, Y² is a protecting group (PG).

In some embodiments, Y² is a suitable hydroxyl protecting group. In some embodiments, Y² is ester protecting group. In some embodiments, Y² is acetate (Ac). In some embodiments, Y² is an isobutanoate (iBu). In some embodiments, Y² is benzoate (Bz).

In some embodiments, Y² is a suitable amine protecting group. In some embodiments, Y² is acetamide (Ac). In some embodiments, Y² is isobutamide (iBu). In some embodiments, Y² is benzamide (Bz). In some embodiments, Y² is =CHN(alkyl)₂. In some embodiments, Y² is =CHN(Me)₂ (dmf). In some embodiments, Y² is

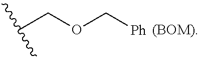 [SMILES: *COCc1ccccc1]

In some embodiments, Y² is a silyl protecting group (e.g., TMS, 2-TES, TES, TIPS, or TBDMS).

In some embodiments, Y² is as depicted in the nucleosides of the Examples section.

As defined above and described herein, Y³ is halogen or —NR₂.

In some embodiments, Y³ is a halogen. In some embodiments, Y³ is —NR₂. In some embodiments, Y³ is a chloro. In some embodiments, Y³ is —N(iPr)₂.

In some embodiments, Y³ is as depicted in the nucleosides of the Examples section.

As defined above and described herein, each Z is independently —O—, —S—, —N(R)—, or —C(R)₂—.

In some embodiments, Z is —O—. In some embodiments, Z is —S—. In some embodiments, Z is —N(R)—. In some embodiments, Z is —C(R)₂—.

In some embodiments, Z is as depicted in the nucleosides of the Examples section.

As defined above and described herein, each n is independently 0, 1, 2, 3, 4, or 5.

In some embodiments, n is 0. In some embodiments, n is 1. In some embodiments, n is 2. In some embodiments, n is 3. In some embodiments, n is 4. In some embodiments, n is 5.

In some embodiments, n is as depicted in the nucleosides of the Examples section.

### EXEMPLIFICATION

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: his (pinacolato)diboron-4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIB: (diacetoxyiodo)benzene
DIBAL-H: diisobutylaluminum hydride
DIPA: diisopropylamine
DIPEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO: dimethyl sulfoxide
DMTr: 4,4′-dimethyoxytrityl
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
LAH: lithium aluminum hydride
LDA: lithium diisopropylamide
LOD limit of detection
L-DBTA: dibenzoyl-L-tartaric acid
m-CPBA: meta-chloroperbenzoic acid
M: molar
ACN: acetonitrile
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
PBS: phosphate buffered saline
PE: petroleum ether
PyBOP: (benzotriazol-1-yloxy)tripyrrolidinophosphonium hexafluorophosphate
Rel: relative
R.T. or rt: room temperature
sat: saturated
SEMCl: chloromethyl-2-trimethylsilylethyl ether
SFC: supercritical fluid chromatography
TBAB: tetrabutylammonium bromide
TBAF: tetrabutylammmonium fluoride
TBAI: tetrabutylammonium iodide
TEA: triethylamine
Tf: trifluoromethanesulfonate
TfAA or Tf₂O: trifluoromethanesulfonic anhydride
TFA: trifluoroacetic acid
TIBSCl: 2,4,6-triisopropylbenzenesulfonyl chloride
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
UPLC: Ultra Performance Liquid Chromatography
wt: weight
Xantphos: 4,5-bis(diphenylphosphino)-9,9-dimethylxanthene

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the nucleic acid or analogues thereof of the present invention are either commercially available or can be produced by organic synthesis methods known to one of ordinary skill in the art (Houben-Weyl 4th Ed. 1952, Methods of Organic Synthesis, Thieme, Volume 21). Further, the nucleic acid or analogues thereof of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain nucleic acid or analogues thereof disclosed herein, one or more ¹H shifts overlap with residual proteo solvent signals; these signals have not been reported in the experimental provided hereinafter.

As depicted in the Examples below, in certain exemplary embodiments, the nucleosides or analogues thereof were prepared according to the following general procedures. It will be appreciated that, although the general methods depict the synthesis of certain nucleic acid or analogues thereof of the present invention, the following general methods, and other methods known to one of ordinary skill in the art, can be applied to all nucleic acid or analogues thereof and subclasses and species of each of these nucleic acid or analogues thereof, as described herein.

### Example 1: Mn-Catalyzed Reactions

Manganese(III) has been reported to effect decarboxylative acetylation of a variety of carboxylic acids in nonaqueous solutions (J. Am. Chem. Soc. 1970, 92, 8, 2450-2460). In presence of AcOH, this reaction reportedly yields corresponding acetate products. The reaction is reported to be even more facile with arylacetic acids with an electron-donating para-substitution on the aromatic ring, or when the carboxylic acid is secondary or tertiary (Chem. Pharm. Bull. 44(12) 2218-2222, 1996; Bioorganic & Medicinal Chemistry Letters 13 (2003) 3433-3435; Bioorganic & Medicinal Chemistry 12 (2004) 903-906). Mn(II) is known to retard the progress of the reaction due to formation of the mixed valence complexes between Mn(II) and Mn(III) (J. Am. Chem. Soc. 1970, 92, 8, 2450-2460).

### Reaction 1: Mn(III) Mediated Decarboxylative Acetylation

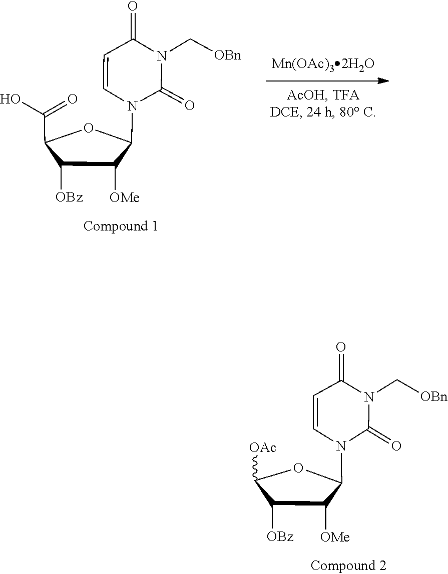

Compound 1 (0.496 g, 1.0 mmol) was dissolved in DCE (5 mL, 10V) under argon. AcOH (0.017 mL, 0.302 mmol) was charged to the reaction mixture with stirring followed by Mn(OAc)₃·2H₂O (1.35 g, 5.04 mmol) and TFA (0.213 mL, 2.76 mmol) at 25° C. After the addition was complete, the reaction mixture was degassed three times with argon, warmed to 80° C. and stirred for 24 h. HPLC analysis after 24 h: 20 μL of the reaction mixture was added in 980 μL of LCMS grade acetonitrile, filtered using a syringe filter and submitted for HPLC analysis.

### Reaction 2: Mn(III) Mediated Decarboxylative Acetylation

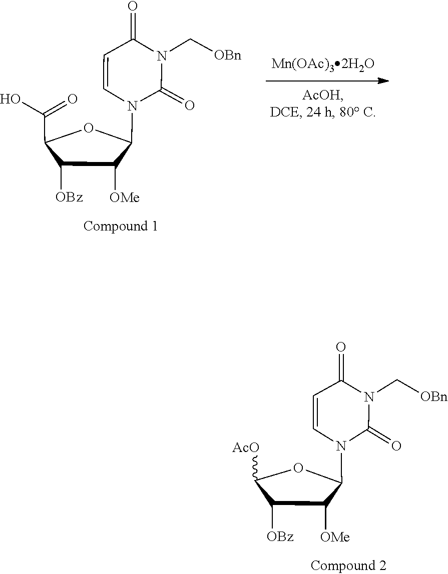

Compound 1 (0.496 g, 1.0 mmol) was dissolved in DCE (5 mL, 10V) under argon. AcOH (0.12 mL, 2.0 mmol) was charged to the reaction mixture with stirring followed by Mn(OAc)₃·2H₂O (0.74 g, 2.76 mmol) at 25° C. After the addition was complete, the reaction mixture was degassed three times with argon, warmed to 80° C. and stirred for 24 h. HPLC analysis after 24 h: 20 μL of the reaction mixture was added in 980 μL of LCMS grade acetonitrile, filtered using a syringe filter and submitted for HPLC analysis.

### Reaction 3: Mn(II) Mediated Decarboxylative Acetylation

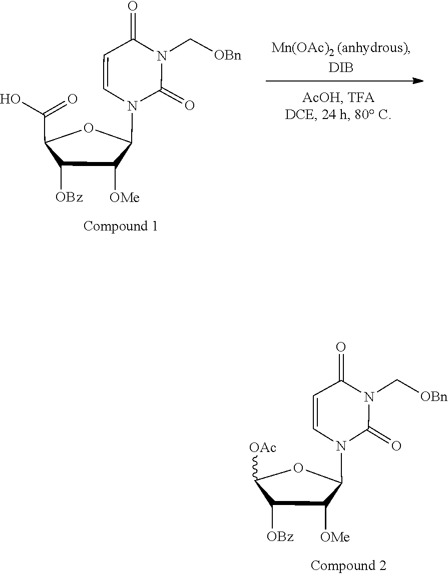

Compound 1 (0.2 g, 0.403 mmol) was dissolved in DCE (2 mL, 10V) under argon. AcOH (7.0 μL, 0.121 mmol) was charged to the reaction mixture with stirring followed by Mn(OAc)₂ (anhydrous) (0.348 g, 2.015 mmol), DIB (0.714 g, 2.217 mmol) and TFA (0.085 mL, 1.112 mmol) at 25° C. After the addition was complete, the reaction mixture was degassed three times with argon, warmed to 80° C. and stirred for 24 h. HPLC analysis after 24 h and 48 h: 20 μL of the reaction mixture was added in 980 μL of LCMS grade acetonitrile, filtered using a syringe filter and submitted for HPLC analysis.

### Reaction 4: Mn(II) Mediated Decarboxylative Acetylation

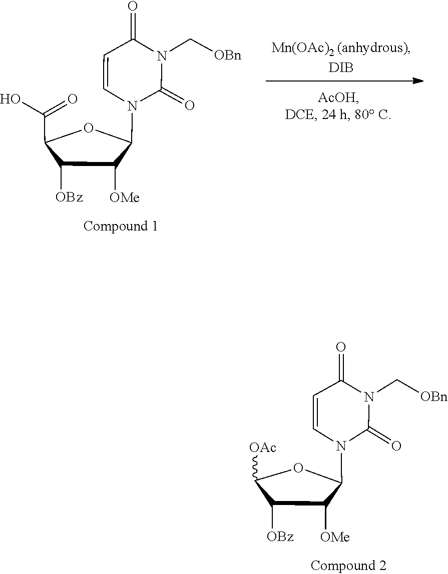

Compound 1 (0.2 g, 0.403 mmol) was dissolved in DCE (2 mL, 10V) under argon. AcOH (0.46 mL, 0.806 mmol) was charged to the reaction mixture with stirring followed by Mn(OAc)₂ (anhydrous) (0.192 g, 1.112 mmol) and DIB (0.394 g, 1.22 mmol) at 25° C. After the addition was complete, the reaction mixture was degassed three times with argon, warmed to 80° C. and stirred for 24 h. HPLC analysis after 24 h and 48 h: 20 μL of the reaction mixture was added in 980 μL of LCMS grade acetonitrile, filtered using a syringe filter and submitted for HPLC analysis.

The HPLC results are shown in the Table 5.

TABLE 5
Mn-Catalyzed Reaction Results

| | | | Mn(OAc)₂ | | | | | | | |
|---|---|---|---|---|---|---|---|---|---|---|
| Rxn | Cmpd | Mn(OAc)₃•2H₂O | Anhyd. | DIB | AcOH | TFA | Time | SM | Product* | |
| # | 1 (g) | Equiv. | Equiv. | Equiv. | Equiv. | Equiv. | (h) | Left | α | β |
| 1 | 0.5 | 5.0 | — | — | 0.3 | 2.76 | 24 | 94.0 | No Product | |
| 2 | 0.5 | 2.76 | — | — | 2.0 | — | 24 | 96.2 | No Product | |
| 3 | 0.2 | — | 5.0 | 5.5 | 0.3 | 2.76 | 24 | 25.8 | 4.5 | 32.9 |
| | | | | | | | 48 | 30.8 | 3.3 | 25.0 |
| 4 | 0.2 | — | 2.76 | 3.036 | 2.0 | — | 24 | 18.8 | 5.4 | 51.4 |
| | | | | | | | 48 | 19.5 | 5.1 | 46.5 |

*α/β product ratio was determined by HPLC integration.

### Example 2: Selected Development Reactions for Mn(II) Mediated Decarboxylative Acetylation

Experiments 3 and 4 showed that using anhydrous Mn(OAc)₂ in presence of DIB yielded product (Compound 2). Experiment 4 without TFA resulted in better conversion to product. In both cases, IPC yield of the product decreased while running the reaction for 48 h.

Experiments 5-10 were performed by use of procedures described for experiment 3 and 4. Reactions varied the equivalence of Mn(OAc)₂, DIB, AcOH, and TFA, while maintaining constant volume of DCE (2 mL). The HPLC results for Experiments 5-10 are shown in Table 6.

TABLE 6
Mn(OAc)₂-Catalyzed Reaction Results

| | | Mn(OAc)₂ | | | | | | | |
|---|---|---|---|---|---|---|---|---|---|
| Rxn | Cmpd | Anhyd. | DIB | AcOH | TFA | Time | SM | Product* | |
| # | 1 (g) | Equiv. | Equiv. | Equiv. | Equiv. | (h) | Left | α | β |
| 5** | 0.2 | 5.0 | 5.5 | 0.3 | 2.76 | 18 | 31.2 | 2.2 | 17.3 |
| 6 | 0.2 | 5.0 | 7.5 | 0.3 | 2.76 | 18 | 25.0 | 3.1 | 23.2 |
| 7 | 0.2 | 5.0 | 7.5 | 2.0 | 1.0 | 18 | 28.8 | 3.1 | 25.8 |
| 8 | 0.2 | 2.76 | 3.04 | 2.0 | — | 18 | 18.2 | 6.6 | 56.6 |
| 9 | 0.2 | 2.76 | 4.14 | 2.0 | — | 18 | 20.1 | 5.0 | 49.4 |
| 10 | 0.2 | 5.0 | 7.5 | 2.0 | — | 18 | 33.5 | 2.4 | 25.1 |

*α/β product ratio was determined by HPLC integration.
**Solvent evaporated during the reaction.

Reactions without TFA produced much better results than the reactions with TFA. Experiments by varying concentration of Mn(OAc)₂ (anhydrous), DIB and AcOH:

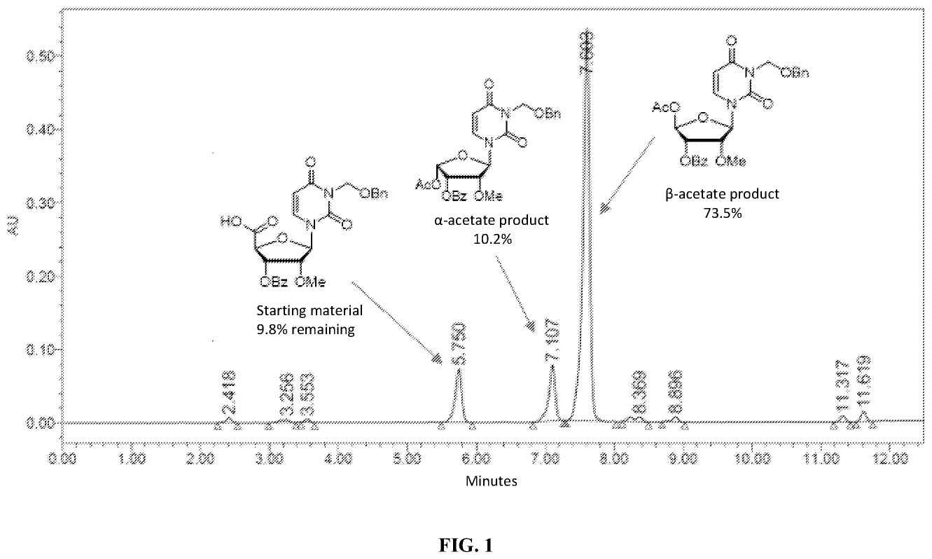

Experiments 11-18 were performed by use of procedures described for experiment 3 and 4. Reactions varied the equivalence of Mn(OAc)₂, DIB, and AcOH while maintaining constant volume of DCE (2 mL). The HPLC results for Experiments 11-18 are shown in the Table 7. FIG. 1 depicts the HPLC chromatogram of the final reaction mixture of reaction #13.

TABLE 7
Mn(OAc)₂-Catalyzed Reaction Results

| | Cmpd 1 | Mn(OAc)₂ | DIB | AcOH | Time | | Product* | |
|---|---|---|---|---|---|---|---|---|
| Rxn # | (g) | Anhyd. Equiv. | Equiv. | Equiv. | (h) | SM Left | α | β |
| 11 | 0.2 | 1.00 | — | 1.00 | 24 | 98.3 | No Product | |
| 12 | 0.2 | 1.00 | 1.10 | 1.00 | 24 | 23.9 | 6.8 | 47.0 |
| 13 | 0.2 | 1.00 | 1.50 | 1.00 | 24 | 9.8 | 10.2 | 73.4 |
| 14 | 0.2 | 1.00 | 0.50 | 1.00 | 24 | 65.2 | 2.4 | 19.6 |
| 15 | 0.2 | 0.50 | 1.00 | 1.00 | 24 | 27.4 | 5.1 | 33.4 |
| 16 | 0.2 | 0.50 | 1.50 | 1.00 | 24 | 13.4 | 8.3 | 52.7 |
| 17 | 0.2 | 2.00 | 2.20 | 1.00 | 24 | 14.2 | 10.0 | 62.4 |
| 18 | 0.2 | 2.00 | 2.20 | 2.00 | 24 | 14.4 | 9.2 | 60.8 |

*α/β product ratio was determined by HPLC integration.

Without DIB, there was no reaction using Mn(OAc)₂ (Experiment 11). With increase in DIB equivalence while using 1 equiv. Mn(OAc)₂, there was significant improvement in product conversion (Experiments 12-14). With an increase in DIB equivalence while using 0.5 equiv. Mn(OAc)₂, there was also significant improvement in product conversion (Experiments 15-16). Using 1 equiv. AcOH produced slightly better result than using 2 equiv. AcOH (Experiments 17-18).

### Example 3: Scale Up Reactions of Mn(II) Mediated Decarboxylative Acetylation to give 4′-O-Acetyl-3′-O-benzoyl-2′-O-methyl-N³-benzyloxymethyluridine

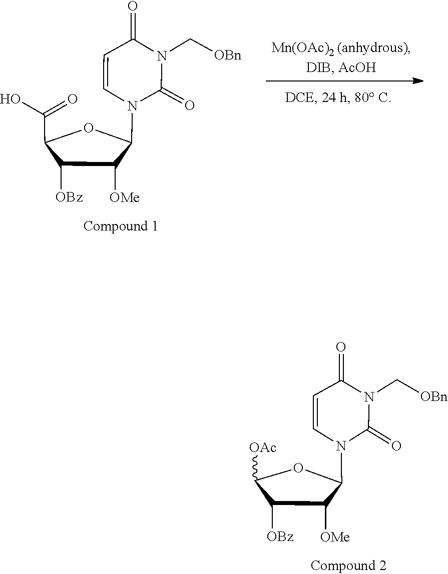

4 mmol Reaction

4′-Carboxy-3′-O-benzoyl-2′-O-methyl-N³-benzyloxymethyluridine (Compound 1) (2.0 g, 4.03 mmol) was dissolved in DCE (20 mL, 10V) under argon. AcOH (0.23 mL, 4.03 mmol) was charged to the reaction mixture with stirring followed by Mn(OAc)₂ (anhydrous) (0.70 g, 4.03 mmol) and DIB (1.94 g, 6.04 mmol) at 25° C. After the addition was complete, the reaction mixture was degassed three times with argon, warmed to 80° C. and stirred for 25 h. The reaction was quenched by aqueous Na₂S₂O₃ (1 M, 40 mL). The reaction mixture was then diluted with ethyl acetate (100 mL) and stirred for 60 min. The dark, black colored reaction mixture was gradually converted to a light, yellow-colored transparent solution. The organic phase was washed with water (3×50 mL), satd. aqueous NaHCO₃ solution (3×50 mL) and brine solution (3×50 mL). The aqueous layer was back-extracted with ethyl acetate (50 mL). The organic layer was combined and dried over Na₂SO₄. The solution was concentrated under reduced pressure to give the crude product, which was purified by column chromatography. Compound 2 was isolated as a white foam (1.46 g, 71.4%).

20 mmol Reaction

4′-Carboxy-3′-O-benzoyl-2′-O-methyl-N³-benzyloxymethyluridine (Compound 1) (10.0 g, 20.16 mmol) was dissolved in DCE (100 mL, 10V) under argon. AcOH (1.16 mL, 20.16 mmol) was charged to the reaction mixture with stirring followed by Mn(OAc)₂ (anhydrous) (3.49 g, 20.16 mmol) and DIB (9.74 g, 30.24 mmol) at 25° C. After the addition was complete, the reaction mixture was degassed three times with argon, warmed to 80° C. and stirred for 25 h. The reaction was quenched by aqueous Na₂S₂O₃ (1 M, 150 mL). The reaction mixture was then diluted with ethyl acetate (250 mL) and stirred for 60 min. The dark, black colored reaction mixture was gradually converted to a light, yellow-colored transparent solution. The organic phase was washed with water (3×100 mL), satd. aqueous NaHCO₃ solution (3×100 mL) and brine solution (3×100 mL). The aqueous layer was back-extracted with ethyl acetate (100 mL). The organic layer was combined and dried over Na₂SO₄. The solution was concentrated under reduced pressure to give the crude product, which was purified by column chromatography. Compound 2 was isolated as a white foam (7.58 g, 73.7%)

### Example 4. Synthesis of 2-cyanoethyl ((2R,3S,4R,5R)-2-((dimethoxyphosphoryl)methoxy)-5-uridyl-4-methoxytetrahydrofuran-3-yl) diisopropylaminophosphoramidite (Compound 10)

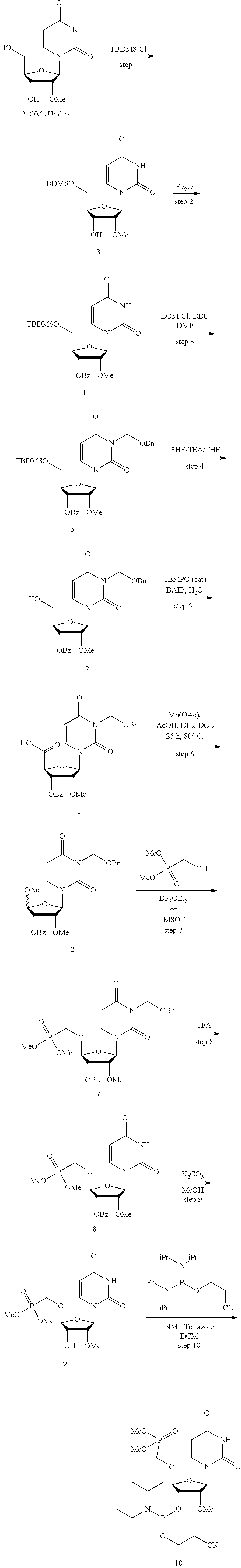

### Synthesis of Compound 3 (5′-O-tert-Butyldimethylsilyl-2′-O-methyluridine)

2′-OMe uridine was dissolved in NMP (5V) and DIPEA (1.6 equiv.). The mixture was cooled to 0° C. (±5° C.) and TBDMS-Cl (1.2 equiv.) was added at a rate to maintain 0° C. (±5° C.). After addition complete, the reaction mixture was warmed to 25° C. (±5° C.) and stirred for an additional 18 h before sampling (SM <5%). The mixture was then added to water (10V) and the aqueous phase extracted with DCM (2×5V). The organic phases were combined and washed successively with water (3V), aq. saturated NaHCO₃, and brine (5V). The resultant organic phase containing Compound 3 was filtered and concentrated to 3V which was used directly in the next step.

### Synthesis of Compound 4 (5′-O-tert-Butyldimethylsilyl-3′-O-benzoyl-2′-O-methyluridine)

The dichloromethane solution of Compound 3 from step 1 was diluted further with DCM (10V). DIPEA (1.3 equiv.) and DMAP (0.1 equiv.) were added to the solution which was then cooled to 5° C. (±5° C.). Benzoic anhydride (1.2 equiv.) was added while maintaining the temperature at 5° C. (±5° C.). The reaction mixture was warmed to 25° C. (±5° C.) and stirred for 15 h before sampling (SM <0.5%). The reaction mixture was washed successively with aq. saturated NaHCO₃ (10V), aq. saturated NaHCO₃ (5V) and water (5V). The organic phase was concentrated under vacuum at 25° C. (±5° C.) to ˜3V. n-Heptane (10V) was added to the solution with vigorous stirring for 4 h. The resulting solids were collected by filtration, washed with n-heptane (1V), then dried under vacuum at 50° C. (±5° C.) for 8-16 h to afford Compound 4 (75% yield over the two steps, LOD ≤5.0%).

### Synthesis of Compound 5 (5′-O-tert-Butyldimethylsilyl-3′-O-benzoyl-2′-O-methyl-N³-benzyloxymethyluridine)

Compound 4 was dissolved in DMF (4V) and DBU (1.5 equiv.) and the solution was cooled to 5° C. (±5° C.) with stirring. BOMCl (1.2 equiv.) was added at a rate to maintain 5° C. (±5° C.). Upon completion of addition, the reaction mixture was brought to 25° C. (±5° C.) and stirred for an additional 5 h before sampling (SM <3%). The reaction mixture was partitioned with EtOAc (7V), and water (10V) cooled to 15° C. (±5° C.). The aqueous phase was back extracted with EtOAc (7V). The organic phases were combined and washed with aq. 15% NaCl solution (2×5V) and concentrated to ˜3V. The concentrate of Compound 5 was co-evaporated with THF (4×5V), each time to 3V, under vacuum at 45° C. (±5° C.). The solution was used directly for the next step.

### Synthesis of Compound 6 (3′-O-Benzoyl-2′-O-methyl-N³-benzyloxymethyluridine)

The THF solution of Compound 5 from step 4 was diluted with THF (5V) and TEA (3.0 equiv.) with stirring. TEA-3HF (2.0 equiv.) was added at 25° C. (±5° C.) and the mixture is allowed to stir for an additional 12 h before sampling (SM <0.5% via HPLC). The reaction mixture was partitioned with MTBE (5V) and water (8V), and the aqueous phase was backextracted with MTBE (5V). The combined organic phases were washed with 0.5N aq. hydrochloric acid (5V), water (3V) and brine (3V). The organic phase was concentrated under vacuum to 3V at 45° C. (±5° C.). The concentrate of Compound 6 was co-evaporated with ACN (2×5V) each time to 2.5V, under vacuum at 45° C. (±5° C.). The resultant solution was used directly for the next step (Compound 8 was formed in 90% yield over the two steps).

### Synthesis of Compound 1 (4′-Carboxy-3′-O-benzoyl-2′-O-methyl-N³-benzyloxymethyluridine)

The ACN solution of Compound 6 from step 4 was further diluted with ACN (1V) and water (4V). TEMPO (0.25 equiv.) was added to the mixture followed by iodobenzene diacetate (2.0 equiv.) at 25° C. (±5° C.). The reaction mixture was stirred for 2 h before sampling (SM <0.5% by HPLC. NaHCO₃ (5 equiv.) was added to the stirring solution and the mixture was concentrated under vacuum @45° C. (±5° C.) to remove all can. NaHCO₃ (1.0 equiv.) was added to the solution, followed by n-heptane (5V), and the mixture stirred for 4 h at 25° C. (±5° C.). The mixture was filtered, and the solids washed with n-heptane (1V). The cake was suspended in ethanol (5V) at 25° C. (±5° C.) and stirred for 4 h. The mixture was filtered, and the solids were washed with ethanol (1V). The solids were dried under vacuum at 50° C. (±5° C.) for 8-16 h to afford Compound 1 (75% yield, LOD ≤5.0%).

### Synthesis of Compound 2 (4′-O-Acetyl-3′-O-benzoyl-2′-O-methyl-N³-benzyloxymethyluridine)

Compound 1 (10.0 g, 20.16 mmol) was dissolved in DCE (100 mL, 10V) under argon. AcOH (1.16 mL, 20.16 mmol) was charged to the reaction mixture with stirring followed by anhydrous Mn(OAc)₂ (3.49 g, 20.16 mmol) and DIB (9.74 g, 30.24 mmol) at 25° C. After the addition was complete, the reaction mixture was degassed three times with argon, warmed to 80° C. and stirred for 25 h. The reaction was quenched by aqueous Na₂S₂O₃ (1 M, 150 mL). The reaction mixture was then diluted with ethyl acetate (250 mL) and stirred for 60 min. The dark, black-colored reaction mixture was gradually converted to a light, yellow-colored transparent solution. The organic phase was washed with water (3×100 mL), satd. aqueous NaHCO₃ solution (3×100 mL) and brine solution (3×100 mL). The aqueous layer was back-extracted with ethyl acetate (100 mL). The organic layer was combined and dried over Na₂SO₄ and concentrated under reduced pressure to give the crude product, which was purified by flash chromatography (silica gel, petroleum ether: ethyl acetate=1:1) or afford Compound 2 as a white foam (7.58 g, 73.7% yield).

### Synthesis of Compound 7 (3′-O-Benzoyl-2′-O-methyl-N³-benzyloxymethyluridine-4′-O-dimethyl (hydroxymethyl)phosphonate

Compound 2 was dissolved in DCM (5V) with stirring under nitrogen. The solution was cooled to 5° C. (±5° C.). BF₃·OEt₂ or TMSOTf (5.0 equiv.) and dimethyl hydroxymethyl phosphonate were added in sequence, slowly while maintaining the temperature at 5° C. (±5° C.). After additions are complete, the mixture was warmed to 25° C. (±5° C.) and stirred for an additional 17 h before sampling (SM ≤5.0% by HPLC). The mixture was cooled to 5° C. (±5° C.) and quenched by addition of water (5V), with stirring for an additional 0.5 h. The organic phase was collected and washed successively with aq. 15% NaCl solution (5V), aq. 1% NaHCO₃ solution (10V) and aq. 15% NaCl solution (5V). The organic phase was concentrated to 1.5-2V, and the residue was purified a silica gel column and eluted with a gradient of 0-70% EtOAc in n-heptane over ˜450V. The fractions were combined (Compound 9 >75% purity) and concentrate at 45° C. (±5° C.) under reduced pressure to 1-1.5V. The solution was co-evaporated with EtOAc (3×IV) and heated to 70° C. (±5° C.) with stirring until all solids dissolved. The solution was cooled to 50° C. (±5° C.) and n-heptane (3.0V) was charged while maintaining temperature. The mixture was cooled to 5° C. (±5° C.) with vigorous agitation and maintained for 0.5 h. Resulting solids were collected by filtration and wash with n-heptane (1V). The re-crystallization was repeated by dissolving solids in EtOAc (1V) at 70° C. (±5° C.), cooling to 50° C. (±5° C.), charging n-heptane (3V) with vigorous stirring, cooling to 5° C. (±5° C.) and maintaining stirring for 0.5 h. The solids were then collected by filtration and washed with n-heptane (1V). The solids were sampled, and recrystallization was repeated until target purity was obtained by HPLC analysis (Compound 7 >90.0% purity, 4′-methoxy impurity ≤1%). The solids were then transferred to a drying oven and subjected to vacuum at 50° C. (±5° C.) for 8-16 h to afford Compound 7 (LOD ≤5.0%).

### Synthesis of Compound 8 (3′-O-Benzoyl-2′-O-methyluridine-4′-O-dimethyl (hydroxymethyl)phosphonate

Compound 7 was added to a solution of stirring trifluoroacetic acid and toluene. The mixture was brought to 40° C. (±5° C.) for 2 h. The solution was sampled, and the reaction was allowed to stir up to 6 h until starting material was consumed (SM ≤3.0% by HPLC). The reaction mixture was partitioned with water (12V) and DCM (8V) at 10° C. (±5° C.), followed by successive washes with aq. 5% NaHCO₃ solution and aq. 15% NaCl solution (5V). The organic phase was concentrated to ˜50% volume (3-5V) at 30° C. (±5° C.) at reduced pressure. Methanol (5.0V) was added to the mixture and the DCM was co-evaporated to 3-5V at 45° C. (±5° C.) at reduced pressure. The solution of Compound 8 was sampled, and the co-evaporation was repeated, if necessary (DCM ≤20%), before use in the next step.

### Synthesis of Compound 9 (2′-O-methyluridine-4′-O-dimethyl (hydroxymethyl)phosphonate

The MeOH solution of Compound 8 from step 8 was diluted further with MeOH to 5V. K₂CO₃ (3.0 equiv.) was added with stirring and the solution was cooled to 15° C. (±5° C.) and stirred for at least 30 min. The reaction mixture was sampled for HPLC analysis (reaction completion when SM <3.0%). The mixture was then filtered, and the cake washed with methanol (1V). The filtrate was cooled to 15° C. (±5° C.) and pH adjusted with acetic acid to pH 6-7. The mixture was concentrated to 2.5-3.0 V at 45° C. (±5° C.). Silica gel (1.5 WT) was added to the solution and the mixture was concentrated to dryness under vacuum while maintaining temperature <50° C. The solids were added to a silica gel chromatography column and eluted successively with n-Heptane (50V), DCM (50V) and 1% MeOH in DCM (50V). The product eluted with a gradient of 1.6-9.0% MeOH in DCM (˜350V). The fractions having Compound 9 (>90% purity by HPLC) were combined and concentrated to 1-2V at 45° C. The concentrate was co-evaporated with ACN (2×2.0V) to bring the final volume to 1-2V. The ACN solution was cooled to 15° C. (±5° C.) and ⅓ of the solution was added to MTBE (15.0V) cooled to 10° C. (±5° C.). The mixture was cooled to 5° C. (±5° C.) remaining ACN solution was added. The mixture is stirred for 2 h, filtered, the solids washed with MTBE (5V), and the solid dried under vacuum at 40° C. (±5° C.) to afford Compound 9 (LOD ≤5.0%).

### Synthesis of Compound 10 (2-cyanoethyl ((2R,3S,4R,5R)-2-((dimethoxyphosphoryl)methoxy)-5-(2,4-dioxo-3,4-dihydropyrimidin-1(2H)-yl)-4-methoxytetrahydrofuran-3-yl) diisopropylaminophosphoramidite)

Compound 9 (1.0 equiv.) was dissolved in DCM (3V) with stirring. In a separate reactor, 2-Cyanoethyl N,N,N′,N′-tetraisopropylphosphorodiamidite (1.3 equiv.), NMI (1.0 equiv.) and tetrazole (0.5 equiv.) were dissolved in DCM (2.0V) and the mixture was cooled to 5° C. (±5° C.). The solution of Compound 9 was added while maintaining 5° C. (±5° C.). The combined mixture was warmed to 20° C. (±5° C.) and stirred for 1.5 h before sampling, followed by sampling every 1 h until SM ≤1.0% (NMT 6 h). The reaction mixture was then washed successively with aqueous saturated NaHCO₃ (5V) and aqueous saturated NaCl (5V). The organic phase was concentrated to 1-1.5V under vacuum at 25° C. (±5° C.). MTBE (3V) was added to the stirring concentrate and cooled to 15° C. (±5° C.). MTBE (12V) as added to the solution and the mixture was cooled to 5° C. (±5° C.) with stirring for 2 h. The resulting solids were collected by filtration, washed with MTBE (1V), and sampled (HPLC ≥94%; P-NMR purity ≥95%; trivalent phosphorous impurities ≤2.0%). Solids were then dried under vacuum at 20° C. (±5° C.) for 8 h. The solids were dissolved in ACN (2V), filtered (0.2 μm filter), and concentrated to IV at 20° C. (±5° C.). MTBE (3V) was added to the stirring concentrate and cooled to 15° C. (±5° C.). MTBE (12V) was added to the solution and the mixture was cooled to 5° C. (±5° C.) with stirring for 2 h. The resulting solids were collected by filtration, washed with MTBE (1V), and dried under vacuum at 20° C. (±5° C.) to afford Compound 10 (residual solvents ≤5.0%, KF≤0.5%).

Prior conditions used to prepare Compound 10 starting from 2′-OMe uridine following the above 10-step synthesis using lead tetraacetate in Step 6 affords Compound 10 with 4.6% overall yield. Using the present invention of using Mn(OAc)₂ in Step 6 and TMSOTf in Step 7, the overall yield of the 10-step process is improved to ˜10% and provides Compound 10 with reduced or no lead impurity (e.g., ≤1 ppm of lead).

### Example 5: Mn²⁺-Catalyzed Reaction on Small Molecule Substrates

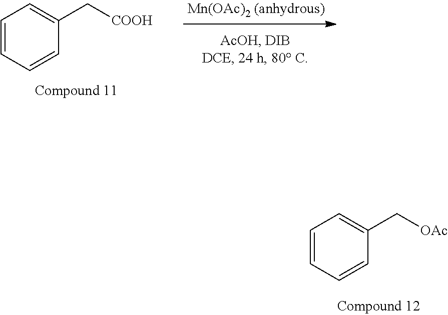 [SMILES: O=C(O)Cc1ccccc1 ; CC(=O)OCc1ccccc1]

### Experimental Procedure

Compound 11 (1.5 g, 11.017 mmol) was dissolved in DCE (15 mL, 10V) under argon. AcOH (0.63 mL, 11.02 mmol) was charged to the reaction mixture with stirring followed by Mn(OAc)₂ (anhydrous) (1.91 g, 11.02 mmol) and DIB (5.32 g, 16.53 mmol) at 25° C. After the addition was complete, the reaction mixture was degassed three times with argon, warmed to 80° C. and stirred for 24 h until the starting material was completely consumed. The reaction was quenched by aqueous Na₂S₂O₃ (1 M, 20 mL) and the reaction mixture was stirred for 30 min. The reaction mixture was then diluted with ethyl acetate (100 mL) and filtered over celite. The filtrate was washed with water (3×20 mL), satd. aqueous NaHCO₃ solution (3×20 mL) and brine solution (3×20 mL). The aqueous layer was backextracted with ethyl acetate (50 mL) and the organic layer was combined and dried over Na₂SO₄. The solution was concentrated under reduced pressure to give the crude product, which was purified by flash column chromatography on silica gel (hexane/ethyl acetate). Compound 12 was isolated as a colorless liquid (1.2 g, 72.7%).

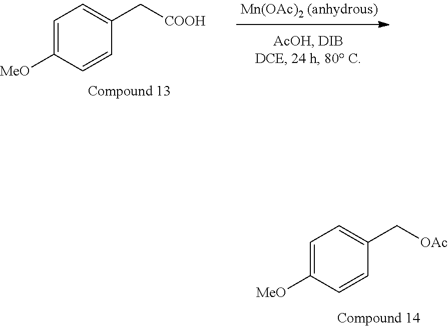 [SMILES: COc1ccc(CC(=O)O)cc1 ; COc1ccc(COC(C)=O)cc1]

### Experimental Procedure

Compound 13 (1.5 g, 9.027 mmol) was dissolved in DCE (15 mL, 10V) under argon. AcOH (0.52 mL, 9.03 mmol) was charged to the reaction mixture with stirring followed by Mn(OAc)₂ (anhydrous) (1.56 g, 9.03 mmol) and DIB (4.36 g, 13.54 mmol) at 25° C. After the addition was complete, the reaction mixture was degassed three times with argon, warmed to 80° C. and stirred for 24 h until the starting material was completely consumed. The reaction was quenched by aqueous Na₂S₂O₃ (1 M, 20 mL) and the reaction mixture was stirred for 30 min. The reaction mixture was then diluted with ethyl acetate (100 mL) and filtered over celite. The filtrate was washed with water (3×20 mL), satd. aqueous NaHCO₃ solution (3×20 mL) and brine solution (3×20 mL). The aqueous layer was backextracted with ethyl acetate (50 mL) and the organic layer was combined and dried over Na₂SO₄. The solution was concentrated under reduced pressure to give the crude product, which was purified by flash column chromatography on silica gel (hexane/ethyl acetate). Compound 14 was isolated as a colorless liquid (1.22 g, 75.3%).

### Example 6. Synthesis of 2-cyanoethyl ((2R,3S,4R,5R)-2-((dimethoxyphosphoryl)methoxy)-5-uridyl-4-methoxytetrahydrofuran-3-yl) diisopropylaminophosphoramidite (Compound 10) using Di-benzoyl Strategy

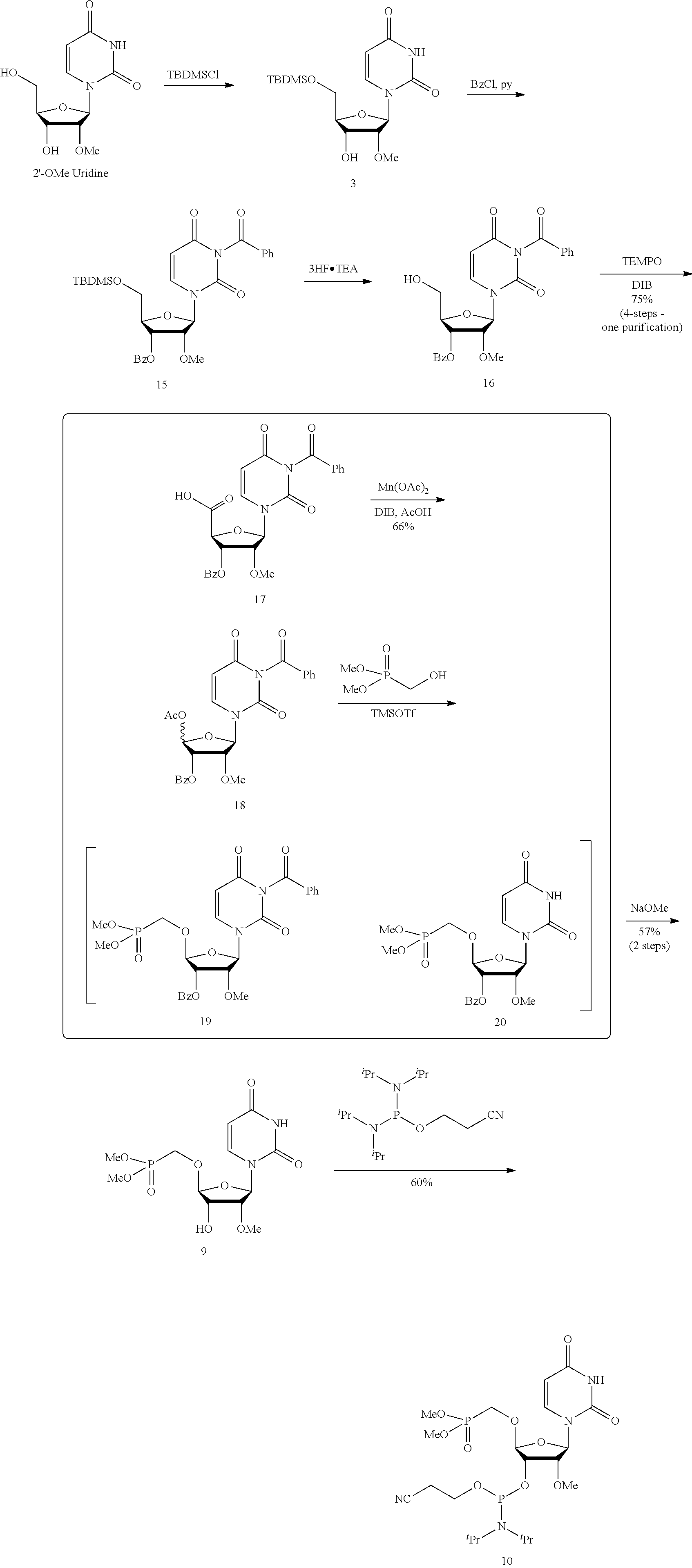

### Synthesis of Compound 3 (5′-O-tert-Butyldimethylsilyl-2′-O-methyluridine)

To a mixture of 2′-OMe uridine (4.0 g, 15.49 mmol) in NMP (20 mL, 5V) was added DIPEA (3.24 mL, 18.59 mmol) at 20° C. The mixture was cooled to 0° C. and TBDMSCl (2.57 g, 17.04 mmol) was added in portion maintaining 0° C. After 17 h, when the starting material was completely consumed, the reaction was quenched by the addition of H₂O (100 mL) at 0° C. with stirring. The mixture was then diluted with ethyl acetate (150 mL), washed with aq. sat. NaHCO₃ (80 mL×2), and brine (80 mL×2). The organic layer was dried over Na₂SO₄, filtered, and concentrated in vacuum to afford Compound 3 as oil. This material was directly used for the next step without any purification.

### Synthesis of Compound 15 (5′-O-tert-Butyldimethylsilyl-3′-O-benzoyl-2′-O-methyl-N³-benzoyluridine)

To a mixture of Compound 3 (15.49 mmol) in pyridine (30 mL) was added DMAP (0.95 g, 7.745 mmol) at 20° C. The mixture was cooled to 0° C. and Benzoyl chloride (7.2 mL, 61.96 mmol) was added while maintaining the temperature at 0° C. The reaction mixture was then warmed to 20° C. and stirred for 6 h. The mixture was again cooled to 0° C. and Benzoyl chloride (3.6 mL, 30.98 mmol) was further added while maintaining the temperature at 0° C. The reaction mixture was then warmed to 20° C. and stirred for again 16 h. The reaction was quenched by the addition of methanol (100 mL) and the reaction mixture was stirred at room temperature for 1 h. The reaction mixture was then evaporated, and the residue was co-evaporated with toluene (100 mL×3) to get rid of the residual pyridine. The residue was then dissolved in ethyl acetate (200 mL) and washed with aq. satd. NaHCO₃ (150 mL), water (150 mL) and brine (150 mL). The organic phase was dried over Na₂SO₄, filtered, concentrated in vacuum to afford Compound 15 as light brown oil. This material was directly used for the next step without any purification.

### Synthesis of Compound 16 (3′-O-benzoyl-2′-O-methyl-N³-benzoyluridine)

To a mixture of Compound 15 (15.49 mmol) in THF (60 mL) was added TEA (6.48 mL, 46.47 mmol). TEA-3HF (5.05 mL, 30.98 mmol) was added to the stirring solution at 20° C. and the mixture was allowed to stir for an additional 17 h. TLC confirmed product formation and complete consumption of Compound 15. Ethyl acetate (200 mL) and water (200 mL) were charged, and the reaction mixture was stirred for 30 min. The organic phase was collected, and the aqueous phase was further extracted with ethyl acetate (150 mL). The organic phases were combined and washed with 0.5N aq. Hydrochloric Acid (200 mL), aq. satd. NaHCO₃ (200 mL) and brine (200 mL). The organic phase was dried over Na₂SO₄, filtered, and concentrated in vacuum to give crude Compound 16. The organic residue was washed with acetonitrile (50 mL×3) and concentrated under vacuum. The residue was directly used for the next step without further purification.

### Synthesis of Compound 17 (4′-Carboxy-3′-O-benzoyl-2′-O-methyl-N³-benzoyluridine)

To a mixture of Compound 16 (15.49 mmol) in ACN (90 mL) and H₂O (60 mL) at room temperature, TEMPO (0.73 g, 4.65 mmol) was charged followed by DIB (9.98 g, 30.98 mmol) portion-wise and the reaction mixture was stirred for 17 h. Progress of the reaction was monitored via TLC until the starting material was almost completely consumed. The reaction mixture was evaporated to dryness. The residue was dissolved in ethyl acetate (300 mL) and washed with water (150 mL×3). The organic phase was dried, concentrated, and purified using column chromatography (DCM/10% MeOH/1% triethylamine). After purification, pure fractions were combined and concentrated to dryness. The residue was dissolved in EtOAc (200 mL). To this, 100 mL 0.4N HCl was added and stirred for 15 min at room temperature. The organic layer was separated and washed with water (150 mL×2) and 10% aq. NaCl solution (50 mL) (if needed, to break the emulsion). The organic phase was then dried over Na₂SO₄ and concentrated. Compound 17 (5.61 g, 75% over 4 steps) was isolated as light-yellow powder.

### Synthesis of Compound 18 (4′-O-Acetyl-3′-O-benzoyl-2′-O-methyl-N³-benzoyluridine)

Compound 17 (1.4 g, 2.917 mmol) in DCE (20 mL) was degassed under argon. AcOH (0.17 mL, 2.917 mmol) was charged to the reaction mixture with stirring followed by Mn(OAc)₂ (anhydrous) (0.504 g, 2.917 mmol) and DIB (1.41 g, 4.37 mmol) at 25° C. After the addition was complete, the reaction mixture was degassed three times with argon, warmed to 80° C. and stirred for 5.5 h. The reaction was quenched by aqueous Na₂S₂O₃ (1 M, 40 mL). The reaction mixture was then diluted with ethyl acetate (100 mL) and stirred for 60 min. The dark, black-colored reaction mixture was gradually converted to a light, yellow-colored transparent solution. The organic phase was washed with water (3×100 mL), satd. aqueous NaHCO₃ solution (3×100 mL) and brine solution (3×100 mL). The aqueous layer was back-extracted with ethyl acetate (100 mL). The organic layer was combined and dried over Na₂SO₄. The solution was concentrated under reduced pressure to give the crude product, which was purified by column chromatography. Compound 18 (0.94 g, 65.3%) was isolated as a crystalline white solid.

### Synthesis of Compound 9 (2′-O-methyluridine-4′-O-dimethyl (hydroxymethyl)phosphonate)

Compound 18 (0.4 g, 0.809 mmol) (dried under high vacuum overnight) and dimethyl hydroxymethyl phosphonate (0.36 mL, 3.236 mmol) were dissolved in dry DCE (4 mL) (4 A molecular sieves) under argon, and the mixture was stirred for 20 min at room temperature. The solution was then cooled to −10° C. TMSOTf (0.44 mL, 2.427 mmol) was added slowly maintaining the temperature. After the addition was complete, the reaction mixture was gradually warmed to room temperature and then heated at 30° C. for 17 h. Formation of Compound 19 and Compound 20 were confirmed via LCMS. The reaction mixture was cooled to 0° C., diluted with ethyl acetate (10 mL), quenched with H₂O (10 mL) and stirred for 15 min. It was further diluted with ethyl acetate (30 mL) and washed with saturated NaHCO₃ (2×30 mL), and brine solution (2×30 mL). The organic phase was dried over Na₂SO₄, concentrated, and the crude mixture was directly used for the next step.

The crude mixture of Compound 19 and 20 (0.809 mmol) was dissolved in MeOH (15 mL) under argon, and to the mixture, NaOMe (30% solution in MeOH) (0.37 mL, 2.0 mmol) was added at room temperature. The reaction was stirred for 1.5 h. Progress of the reaction was checked using HPLC which showed complete consumption of the starting materials after 1.5 h. The reaction mixture was neutralized with AcOH (4 drops) and concentrated. The crude reaction products were dissolved with ethyl acetate (30 mL) and washed with saturated NaHCO₃ (2×30 mL), and brine solution (2×30 mL). The organic phase was dried over Na₂SO₄, concentrated, and the crude mixture was purified using column chromatography to afford Compound 9 (0.168 g, 57% over 2 steps) as a white foam.

### Synthesis of Compound 10 (2-cyanoethyl ((2R,3S,4R,5R)-2-((dimethoxyphosphoryl)methoxy)-5-(2,4-dioxo-3,4-dihydropyrimidin-1(2H)-yl)-4-methoxytetrahydrofuran-3-yl) diisopropylaminophosphoramidite)

Synthesis of Compound 10 was mentioned in Example 4.

By implementing this above-mentioned di-benzoyl strategy, Compound 10 starting from 2′-OMe uridine could be synthesized in 7 steps with overall ˜15-18% expected yield.

While several embodiments of this invention have been described herein, it is apparent that the basic examples provided herein may be altered to provide other embodiments that utilize the nucleic acid or analogues thereof and methods of this invention. Therefore, it will be appreciated that the scope of this invention is to be defined by the specification and appended claims rather than by the specific embodiments that have been represented by way of example.

1. A process for preparing a compound comprising an acetoxy group, wherein the compound comprising an acetoxy group is represented by formula B: 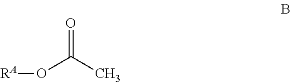
or salt thereof, comprising the steps:
(a) providing a compound comprising a carboxyl group represented by formula A: 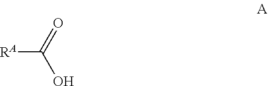
or salt or ester thereof, and
(b) subjecting the compound of formula A to conditions sufficient to form the compound of formula B, wherein the conditions comprise a manganese(II) reagent and an oxidizing agent, or wherein the conditions comprise a manganese(III) reagent, and wherein:
R^{A} is an optionally substituted group selected from alkyl, aryl, heteroaryl, carbocyclyl, heterocyclyl, protected amino acid, protected nucleoside, protected nucleotide, and protected oligonucleotide, wherein each of aryl and heteroaryl is independently monocyclic or bicyclic and each of carbocyclyl and heterocyclyl is independently monocyclic, bicyclic, bridged bicyclic, or spirocyclic.

2. A process for preparing a nucleoside or analogue thereof comprising a 4′-acetoxy group, wherein the nucleoside or analogue thereof comprising a 4′-acetoxy group is represented by formula I-b: 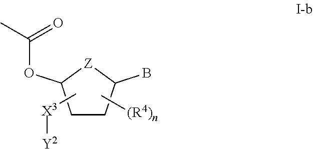
or a salt thereof, comprising the steps:
(a) providing a nucleoside or analogue thereof comprising a 4′-carboxyl group represented by formula I-a: 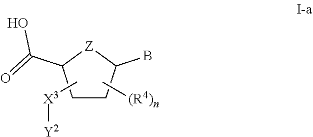
or a salt or ester thereof, and
(b) subjecting the nucleoside or analogue thereof of formula I-a to conditions sufficient to form the nucleoside or analogue thereof of formula I-b,
wherein the conditions comprise a manganese(II) reagent and an oxidizing agent, or wherein the conditions comprise a manganese(III) reagent, and wherein:
each B is independently a nucleobase or hydrogen;
each R⁴ is independently hydrogen, fluoro, —OH, —OC₁₋₆alkyl, —OCH₂CH₂OC₁₋₆alkyl, or —O-protecting group (—OPG);
each X³ is independently —O—, —S—, or —N(R)—;
each R is independently hydrogen, a protecting group (PG), or an optionally substituted group selected from C₁₋₆ aliphatic, phenyl, a 4-7 membered saturated or partially unsaturated carbocyclic or heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur;
each Y² is independently hydrogen or a protecting group (PG);
each Z is independently —O—, —S—, —N(R)—, or —C(R)₂—; and
each n is independently 0, 1, 2, 3, 4, or 5.

3. The process of claim 1, wherein the manganese(II) reagent is Mn(OAc)₂.

4. The process of claim 1, wherein the manganese(II) reagent is anhydrous Mn(OAc)₂.

5. The process of claim 1, wherein the conditions further comprises an oxidizing reagent.

6. The process of claim 5, wherein the oxidizing reagent is (diacetoxyiodo)benzene (DIB).

7. The process of claim 1, wherein the conditions further comprises an acid.

8. The process of claim 7, wherein the acid is acetic acid.

9. The process of claim 1, wherein the conditions further comprises a solvent.

10. The process of claim 9, wherein the solvent is 1,2-dichloroethane (DCE).

11. The process of claim 1, wherein the conditions further comprise heating the reaction mixture to about 20-100° C. for about 2-48 hours.

12. The process of claim 1, wherein the conditions further comprise heating the reaction mixture to about 80° C. for about 5 hours.

13. The process of claim 2, wherein the nucleoside or analogue thereof of formula I-b is a nucleoside or analogue thereof of formula I-b-1: 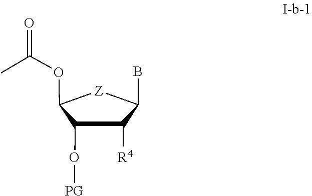
or a salt thereof.

14. The process of claim 2, further comprising the steps of preparing a nucleotide or analogue of formula I-d: 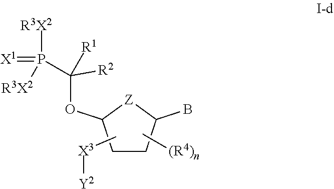
or a salt thereof, comprising the steps:
(a) providing a nucleoside or analogue thereof of formula I-b: 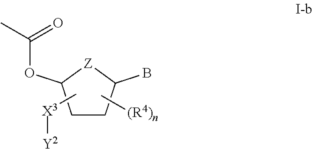
or a salt thereof, and
(b) reacting the nucleoside or analogue thereof of formula I-b with a compound of formula I-c: 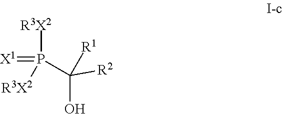
to form the nucleotide or analogue thereof of formula I-d, wherein:
each B is a nucleobase or hydrogen;
R¹ and R² are independently hydrogen or C₁₋₆alkyl;
each R³ is independently hydrogen, a protecting group (PG), a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁴ is independently hydrogen, fluoro, —OH, —OC₁₋₆alkyl, —OCH₂CH₂OC₁₋₆alkyl, or —O-protecting group (—OPG);
X¹ is O, S, or NR;
each R is independently hydrogen, a protecting group (PG), or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur;
each X² is independently —O—, —S—, —B(H)₂—, or a covalent bond;
X³ is —O—, —S—, or —N(R)—;
Y² is hydrogen or a protecting group (PG);
each Z is independently —O—, —S—, —N(R)—, or —C(R)₂—; and
each n is independently 0, 1, 2, 3, 4, or 5.

15. The process of claim 14, wherein the nucleotide or analogue thereof of formula I-d is a nucleotide or analogue thereof of formula I-d-1: 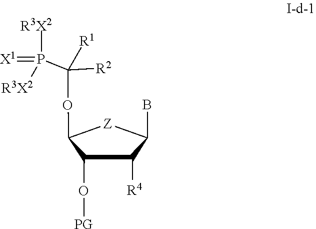
or a salt thereof.

16. The process of claim 14, further comprising the steps of preparing a nucleotide or analogue of formula I-e: 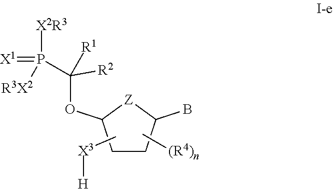
or a salt thereof, comprising the steps:
(a) providing a nucleotide or analogue thereof of formula I-d: 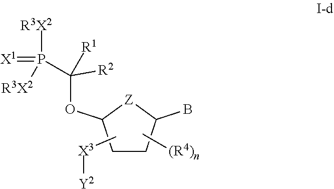
or a salt thereof, and
(b) deprotecting the nucleotide or analogue thereof of formula I-d to form the nucleotide or analogue thereof of formula I-e.

17. The process of claim 16, wherein the nucleotide or analogue thereof of formula I-e is a nucleotide or analogue thereof of formula I-e-1: 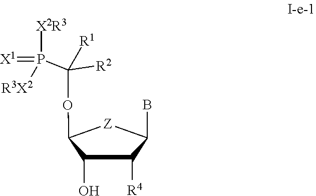
or a salt thereof.

18. The process of claim 16, further comprising the steps of preparing a nucleotide or analogue of formula I-g: 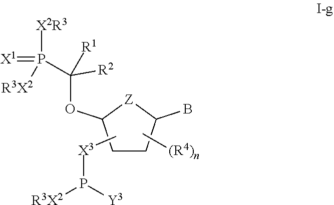
or a salt thereof, comprising the steps:
(a) providing a nucleotide or analogue thereof of formula I-e: 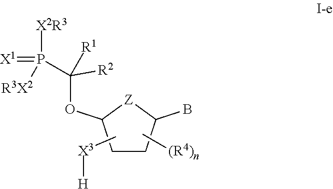
or a salt thereof, and
(b) reacting the nucleotide or analogue thereof of formula I-e with a compound of formula I-f: 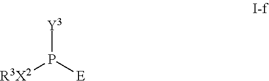
to form the nucleotide or analogue thereof of formula I-g, wherein:
E is halogen or —NR₂; and
Y³ is halogen or —NR₂.

19. The process of claim 18, wherein the nucleotide or analogue thereof of formula I-g is a nucleotide or analogue thereof of formula I-g-1: 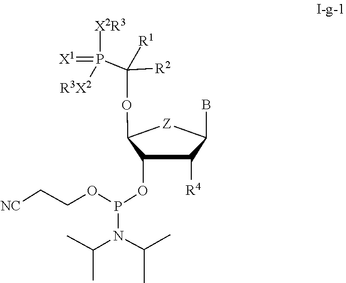
or a salt thereof.

20. The process of claim 2, wherein PG is an ester protecting group.

21. The process of claim 2, wherein PG is benzoate.

22. The process of claim 14, wherein R¹ is hydrogen and R² is hydrogen or methyl.

23. The process of claim 2, wherein n is 1 and R⁴ is hydrogen, fluoro, —OH, —OMe, or —OCH₂CH₂OMe.

24. The process of claim 2, wherein B is a nucleobase.

25. The process of claim 24, wherein the nucleobase is a protected nucleobase.

26. The process of claim 2, wherein each B is selected from

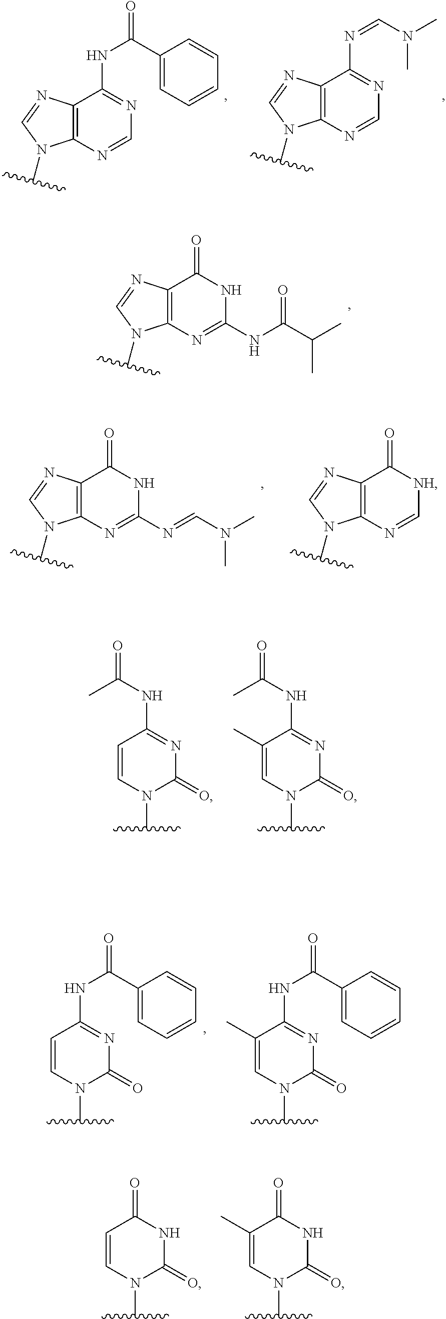

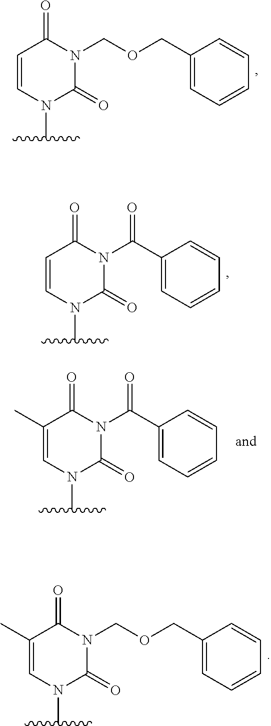

27. A process for preparing a nucleoside or analogue thereof comprising a 4′-acetoxy group, wherein the nucleoside or analogue thereof comprising a 4′-acetoxy group is represented by formula I-b-1: 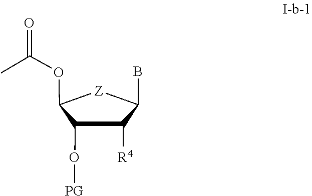
or a pharmaceutically acceptable salt thereof, comprising the steps:
(a) providing a nucleoside or analogue thereof comprising a 4′-carboxyl group represented by formula I-a-1: 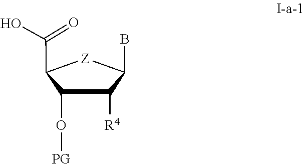
or a pharmaceutically acceptable salt or ester thereof, and
(b) subjecting the nucleoside or analogue thereof of formula I-a-1 to conditions sufficient to form the nucleoside or analogue thereof of formula I-b-1,
wherein the conditions sufficient to form the nucleoside or analogue thereof of formula I-b-1 comprise the steps:
(i) combining the nucleoside or analogue thereof of formula I-a-1 with dichloroethane (DCE) to form a mixture;
(ii) adding AcOH to the mixture of step (a) with agitation;
(ii) adding Mn(OAc)₂ and (diacetoxyiodo)benzene) (DIB) to the agitated mixture of step (b); and
(iv) heating the mixture of step (c) at about 80° C. for about 5 hours, wherein:
each B is independently a nucleobase;
each PG is a protecting group;
each R⁴ is independently hydrogen, fluoro, —OH, —OC₁₋₆alkyl, —OCH₂CH₂OC₁₋₆alkyl, or —O-protecting group (—OPG);
each Z is independently —O—, —S—, —N(R)—, or —C(R)₂—; and
each R is independently hydrogen, a protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form a 4-7 membered saturated or partially unsaturated carbocyclic or heterocyclic ring having 0-3 heteroatoms, independently selected from nitrogen, oxygen, and sulfur.
